(19) United States
(12) Patent Application Publication
(10) Pub. No.: US 2018/0228552 A1
(43) Pub. Date: Aug. 16, 2018

(54) SURGICAL CELL, BIOLOGICS AND DRUG DEPOSITION IN VIVO, AND REAL-TIME TISSUE MODIFICATION WITH TOMOGRAPHIC IMAGE GUIDANCE AND METHODS OF USE

(71) Applicant: THE BOARD OF REGENTS, THE UNIVERSITY OF TEXAS SYSTEM, Austin, TX (US)

(72) Inventors: Thomas E. MILNER, Elgin, TX (US); Janet ZOLDAN, Austin, TX (US); R.Y. Declan FLEMING, Austin, TX (US); Nitesh KATTA, Austin, TX (US); John RECTOR, Austin, TX (US); Michael R. GARDNER, Austin, TX (US); Arnold ESTRADA, Austin, TX (US); Austin MCELROY, Austin, TX (US); Marc D. FELDMAN, Austin, TX (US)

(21) Appl. No.: 15/883,963

(22) Filed: Jan. 30, 2018

Related U.S. Application Data
(60) Provisional application No. 62/452,186, filed on Jan. 30, 2017.

(51) Int. Cl. A61B 34/20; A61B 90/00; A61B 5/00; A61B 3/10

(52) U.S. Cl. CPC A61B 34/20; A61B 90/37; A61B 5/0066; A61F 2009/00851; A61B 2090/3735; A61B 2034/2055; A61B 2090/364; A61B 3/102

(57) ABSTRACT

Provided herein are systems, methods and apparatuses for an in vivo surgical device that uses tomographic imaging to guide the process of surgical incisions for cell, biologics and drug delivery; the image guided system guides the process of delivery with comprehensive real-time processing with the ability to seal the location of delivery and offer laser-tissue modification via a co-aligned tissue modification beam on tissue without tissue damage to adjacent critical or delicate structures.

### CROSS-REFERENCE TO RELATED APPLICATIONS

The present application claims priority to U.S. provisional application Ser. No. 62/452,186, filed Jan. 30, 2017, herein incorporated by reference in its entirety.

### BACKGROUND

The invention generally relates to imaging and surgery.

Cell, biologic and drug delivery to specific locations in the body cavity has always been crude and based on surgeon's ability and experience with the human anatomy like in the case of cartilage treatment in osteoarthritis or therapeutic treatment post-surgical resection of tumors in cancer surgeries. Currently, intrasurgical cell, biologic and drug deliveries are performed by an expert doctor through the use of a laparoscopes, wide field imaging, to provide a diagnosis and guide surgical injection. This has many drawbacks that cause the surgery and delivery to be unreliable and subjective.

The American Cancer Society's estimate for the incidence of malignant brain and spinal cord tumors in the United States for 2015 is about 23,770 (13,350 in males and 10,420 in females). These numbers would be significantly higher if benign tumors were also included. Complete surgical resection of these tumors remains the standard protocol for almost all a priori resectable tumors as defined by preoperative standard computed tomography (CT).

Current state-of-the-art techniques for tumor resection (e.g., NICO Myriad, Medtronic StealthStation, Zeiss OPMI) employ techniques including iMRI, iCT, fluoroscopy and preoperative CT/MRI to provide the surgeon image information on the location and a navigable path to the tumor. Other state-of-the-art surgical techniques use intra-operative ultrasound with prior knowledge from MRI and CT. Although these imaging techniques provide a wide field image, recorded images have limited resolution (>100 μm) and preoperative imaging primarily provides information on the location and a pathway to access the tumor. For cases like iMRT, the entire surgical theatre needs to be reconfigured (plastic surgical tools) to fully utilize MRI images during surgery. Although fluorescence imaging can offer higher resolution (micron/sub-micron) the imaging is confined to the tissue surface and identifying locations of sub-surface delicate structures remains problematic. Pathologist recommendation on resected tissues remains the gold standard for surgical margin assessment, resulting in extended time duration of surgery and associated anesthesia. Considering these limitations, Optical Coherence Tomography (OCT) occupies a useful niche in the resolution vs. imaging depth trade-off. Plaque classification is an example of the benefits realized using intravascular OCT compared to IVUS (intravascular Ultra Sound). Thus, employing a surgical tool guided by OCT may offer a more effective resection of tissue or tumors positioned near delicate tissue structures that should not be damaged.

The present invention solves these problems as well as others.

### SUMMARY OF THE INVENTION

Provided herein are systems, methods and apparatuses for an in vivo surgical device that uses tomographic imaging to guide the process of surgical incisions for cell, biologics and drug delivery; the information the image-guided system records guides the process of delivery with comprehensive real-time processing with the ability to seal the location of delivery and offer laser-tissue modification via a co-aligned tissue modification beam on tissue without tissue damage to adjacent critical or delicate structures.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1a is a schematic for a work flow for image guided system; FIG. 1b is a schematic of the data flow and representation in the image guided system; FIG. 1c are tomographic images for different scan dimensions; and FIG. 1d is a flow chart for the image guided system incorporated in multiple surgical scenarios.

FIG. 2a is a schematic overview of the image guide system with co-aligned laser and OCT beams; FIG. 2b is design of handheld interface; FIG. 2c is a schematic of a forward looking cutting laser coupled with a side cutting laser.

FIGS. 3a-3b are OCT images showing versatility in the formation of the microwells by selecting a line to create it, where FIGS. 3a, 3b are enface images of the phantom before and after the formation of the microwells, and FIG. 3c is the cross-section image of the microwell, and the scale bars are 200 μm.

FIGS. 4a-4b are a time-lapse OCT Imaging as the cutting laser is creating microwells in the tissue phantom at different periods of time, where FIG. 4a is at 0 sec and FIG. 4b is at 3 sec. The white arrow 101 highlights the OCT imaging the tissue material as it blows off the tissue, the scale bars are 200 μm

FIGS. 5a-5d are automated OCT Image guidance to control the laser beam position and laser dosimetry to cut around structures OCT versatility showcased in the creation of cutting sites while automatically avoiding structures (in this case the micro-vessel on the surface), where FIGS. 5a, 5b are enface images of the phantom before and after the formation cutting with the laser, and

FIGS. 5c, 5d are the cross-section image of the phantom, the scale bars are 200 μm.

FIGS. 6a-6c are time-lapse OCT images showcasing the image guidance versatility in the form of depositing material into a created microwell site at different period of time, where FIG. 6a is at 0 sec, FIG. 6b is at 1.5 sec, and FIG. 6c is at 3 seconds, the scale bars are 200 μm.

FIGS. 7a-7b is a thickness mapping performed on single OCT b-scan, where FIG. 7a is a left image displays the b-scan with the cartilage/bone boundary traced in green, and FIG. 7b is a right plot displays the thickness values corresponding to each a-scan of the b-scan.

FIGS. 8a-8c is Cartilage metrics for Region 1. Scale bars are 1 mm, where FIG. 8a is the attention coefficient; FIG. 8b is the thickness measurements in microns; and FIG. 8c is the gradient measurements in degrees.

FIGS. 9a-9c is Cartilage metrics for Region 2. Scale bars are 1 mm, where FIG. 9a is the attention coefficient; FIG. 9b is the thickness measurements in microns; and FIG. 9c is the gradient measurements in degrees.

FIGS. 10a-10c is Cartilage metrics for Region 3. Scale bars are 1 mm, where FIG. 10a is the attention coefficient; FIG. 10b is the thickness measurements in microns; and FIG. 10c is the gradient measurements in degrees.

FIG. 11 is lateral beam profile of the laser at the focal spot of the image guided system.

FIGS. 12a-12c are OCT images illustrating the line-cuts (1 mm and 400 μm) made using the image guided system. FIGS. 12a-12b images show the before and after OCT images of the tissue phantom with the highlighted (in black line) cut in the tissue. The white-dotted-line highlighted image shows the cross-section OCT image at the location, as shown in FIG. 12c.

FIGS. 13a-13d are enface and cross-section images obtained from OCT imaging for cutting up to a blood vessel, where FIG. 13a is an enface image of the blood vessel going deeper into the tissue phantom; FIG. 13b is an enface image after the laser cut; and FIGS. 13c and 13d are cross section images of the vessel before and after cutting; and scale bars are 200 μm.

FIG. 14 is a graph of the computed flux along the z-distance (depth into the tissue in millimeters).

FIG. 15a is a simulation model for laser cuts alongside the pneumatic experimental setup; FIG. 15b is a finite element modeling to compute Arrhenius damage; and FIG. 15c are two-photon enface image and cross-sectional images for two different focal depths, where the results overlaid on the predicted etch depths from the blow off model and the Removal rate, R is estimated for a given laser power.

FIG. 16 is a graph of the volumetric tissue removal rate experimental in comparison to the modeled value.

FIG. 17a is an OCT image of the attenuation coefficient; and FIG. 17b is the flow angiogram.

FIG. 18a shows the attenuation+flow overlay and FIG. 18b shows the fluorescence comparison.

FIGS. 19a-19h are OCT images demonstrating coagulation.

FIGS. 20a-20d are OCT images demonstrating coagulation in a mouse brain in vivo.

FIG. 21a is a y-z OCT image before skin being cut, and FIG. 21b is a y-z OCT image after the skin has been cut. FIG. 21c is an x-z OCT image before being cut, and FIG. 21d is an x-z OCT image after the skin has been cut.

FIG. 22 is a graph showing the OCT results verified with a PDMS sample and an IR camera.

FIG. 23a is an OCT Image of Occluded Artery Before Ablation; FIG. 23b is an OCT Image of Occluded Artery After Ablation, where the square highlights the Ablated region showing Unablated calcium nodules; and FIG. 23c is an Histology Image of Occluded Artery Before Ablation.

FIG. 24a is an OCT image of the cartilage after 1 Tm laser sweep. FIG. 24b is an OCT image of the cartilage after multiple Tm laser sweeps showing the micropore. And FIG. 24c is an OCT image of the cartilage after 100 Tm laser passes showing the increased diameter of the micropore.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

An image-guided system for precision cell, biologics and drug deposition is disclosed. In one embodiment, the image-guided system comprises a multi-lumen surgical probe that allows in vivo, real-time coherence tomography (like optical coherence tomography (OCT) or polarization sensitive OCT, here after we refer to these techniques as OCT to encompass the different coherence tomography techniques) imaging analysis of biological tissues, surface modification of the tissue with a co-aligned cutting laser, and subsequent deposition of biologic/cell/therapeutic material into and on the tissue surface. The image-guided system and device comprises an OCT imaging probe that provides information for guidance on the cutting depth, location of delicate structures (e.g., blood vessels, nerves, etc.) and differentiation or classification of different types of tissue. The image-guided system and device comprises a co-aligned tissue modifying laser that will make micro cuts in the tissue, modifying the tissue for increased efficacy of therapeutic materials. In one embodiment, the tissue modifying laser creates micro-wells viable for cell deposition; alternatively, the tissue modifying laser coagulates or assists in a chromophore-assisted laser inactivation process or a photo chemical modification. The image-guided system and device further comprises a deposition tool interfaced with the co-aligned OCT/tissue-modifying-laser probe injects cells, biologics or drugs into the location using OCT to decide on which to inject and tomographic imaging to guide the process of injecting the material. The biological tissue and material can range from autologous stem cells, to chemotherapeutics, to hydrogel scaffolds to other drugs/biomaterials. The device features an integrated system that delivers controlled volumes of these cells, biologics or therapeutic materials to the location, which are all guided with the OCT feedback. The image-guided system and device includes a precision at the micro-scale for sub-surface imaging, tissue removal, and volume dispensation. While a laser system has been described for cutting tissue, other cutting systems may be used to cut tissue, such as standard surgical tools or tissue modifying tools.

Micro-cuts may comprise an incision into a tissue between about 1 μm and about 1000 μm. A micro-well may comprise an incision into a tissue between about 1 μm and about 1000 μm with a depth between about 1 μm and about 1000 μm.

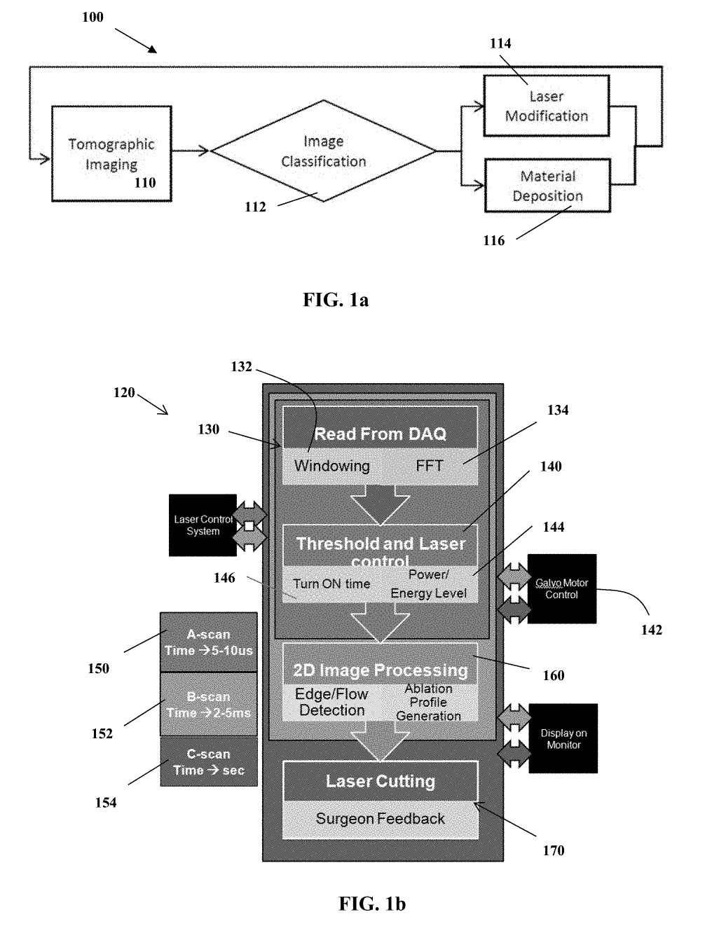

As shown in FIG. 1a, the image-guided system and method 100 starts with a tomographic imaging step 110. The coherence tomography information obtained from the tomographic imaging step 110 is used to classify/differentiate tissue in an Image Classification step 112. The image-guided system bifurcates into two possibilities after this Image Classification step 112. Either 1) the Image Classification step 112 informs the surgeon about the site for injection of the material or material deposition 116 (e.g. cell, biologic or drug), which may then deposit the material; or 2) the Image Classification step 112 informs an appropriate laser modification 114 of the tissue to be carried out based on the classification information. The laser modification 114 of the tissue includes either an ablation or a coagulation or chromophore-assisted laser inactivation or a photo chemical modification. In one embodiment, the laser modification step will include one or more of the following: a change the laser pulse energy, a change the laser pulse repetition rate, a change the laser pulse duration, a change the spot size of the laser beam on the tissue; and/or the path and velocity of the beam on the tissue. The laser modification changes may be operable to complete coagulation or ablation. Subsequently, the image guided system repeats until the ascribed surgical objective of depositing cells and/or biologics and/or drug material is completed.

Data Flow for OCT Image Guidance:

The image guided system and method may include a data flow for OCT image guidance 120, as shown in FIG. 1b. The image guided system and method comprises computing an OCT image. The OCT image is computed using standard OCT processing techniques 130 comprising acquiring the spectral fringe signal to two-byte values, applying a window (e.g., Hanning) 132, computing a fast Fourier transform 134 and a resultant power spectrum vs. time delay of light propagating into the sample or tissue. The three-dimensional OCT image information could be used in a variety of ways to guide the laser ablation 140 or other surgical procedure. In one embodiment, OCT imaging may be used to control at what lateral or longitudinal positions the ablation laser is enabled by a scanning mirror such as a GalvoMotor control 142, and with what average power or energy level 144. To accomplish this, profiles of laser turn-ON and turn-OFF times 146 that avoided simulated subsurface vessels are generated and stored in an XML file based on the OCT images. Finally, the operator or examination system (e.g., surgeon) approved the proposed ablation pattern 170 to initiate tissue removal. The system reads the XML file and turned on the cutting laser at the appropriate A-scan locations 150 during imaging of the next frame so as to avoid detected structures through 2D image processing 160. A single depth profile (Intensity vs Depth) is called an A-Scan. The 2D image processing 160 may include Edge/Flow detection and ablation profile generation. FIGS. 1b-1c illustrates the time scale for different scan dimensions and general data flow in the image guided system.

The image guided system and method may generally comprise OCT imaging and construction of contrast images and feature detection including angiography, tissue optical properties, thermography, attenuation, and the like; generating a coagulation pattern or tissue regions targeted for coagulation by using the contrast image and completing the feature selection; generating a first laser dosimetry for coagulation; performing coagulation with the laser; OCT images after coagulation or cutting to generate contrast images; detecting features of damaged and undamaged tissue; updating the ablation pattern; generating a second laser dosimetry for ablation if necessary; performing ablation; and OCT imaging after cutting/ablation and generating contrast images. This system and method may be repeated as necessary to initiate the next step in coagulation and ablation/cutting.

The generation of the coagulation pattern for the first dosimetry generator is the signals applied to the scanning system and the laser that will result in the coagulation of the tissue. At least two types of control signals are generated in this step: 1) scanning control signals; 2) a first laser dosimetry signals. The former include at least two signals for x- and y-positioning of the laser beam. The first laser dosimetry signals include: laser pulse energy; laser pulse duration; laser pulse repetition frequency; laser spot size; laser wavelength. The coagulation parameters can vary depending on whether a tissue region is vascular or avascular. The OCT imaging after coagulation and generation of contrast images is important since if the vasculature is not coagulated then OCT imaging and feature detection may need to be repeated until the targeted tissue regions are completely coagulated. This process in completed until the coagulation step is complete.

The generation of an ablation pattern for thus second dosimetry generator is similar to generation of the coagulation pattern for the first dosimetry generator only for tissue cutting and ablation. At least two types of control signals are generated in this step: 1) scanning control signals; 2) second laser dosimetry signals. The former include at least two signals for x- and y-positioning of the laser beam. The laser dosimetry signals include: laser pulse energy; laser pulse duration; laser pulse repetition frequency; laser spot size; laser wavelength; the coagulation parameters can vary depending on whether a tissue region is vascular or avascular.

The OCT imaging after cutting/ablation and generating contrast images step is important since if the vasculature is not coagulated then OCT imaging after coagulation and generation of contrast images, generation of the ablation pattern and second dosimetry, and tissue cutting/ablation may need to be repeated until the targeted tissue regions are completely cut or ablated. This process in completed until the cutting/ablation step is complete.

Overview of Image Guided System

As shown in FIG. 1d, the image guided system allows surgeons to more effectively perform surgery of diseased tissues by integrating Optical Coherence Tomography (OCT) imaging with the laser treatment device or any surgical procedure. OCT provides rapid, high-resolution, three-dimensional image information that can provide valuable feedback to produce better treatment outcomes. The OCT imaging information may be utilized in several modes. In one embodiment, the OCT image information of the diseased area and surrounding tissue will be presented to the surgeon as a guide towards identification of diseased vs. normal tissue sites and more precise treatment with the ability to minimize non-specific damage to adjacent tissues. For example, OCT information can be used to record angiography images that provide a map of the vasculature in the tissue. Vascular geometry combined with optical attenuation can indicate regions of abnormal tissue such as a tumor. In another embodiment, the OCT image information will be used for real-time control of laser dosimetry or robotic cutting or any surgical procedure. Laser dosimetry includes pulse energy, laser pulse duration, pulse repetition rate, spot size on the tissue and laser emission wavelength. In this mode, the OCT image information will be used with rapid control algorithms to minimize non-specific damage and need not be explicitly presented to the surgeon.

The image guided system comprises a Combined Holistic Surgical View subsystem 180, a Feature Detection Image Overlay subsystem 182, an examination 184, a Positioning subsystem 186, and a Surgeon Initiated Laser Treatment 188. The Combined Holistic Surgical View subsystem 180 is where preoperative imaging and intraoperative imaging are incorporated and combined into one holistic view of the surgical field. High resolution volume OCT images can be added to this view as the surgeon acquires images intraoperatively with the smart laser probe.

The Feature Detection Image Overlay subsystem 182 is where OCT volume images are analyzed and features of surgical relevance (vascular geometry, tissue optical properties, or tissue composition (e.g., lipid vs. water)) are highlighted and overlaid on the holistic view presented to the surgeon described above. Then, in one embodiment, the examination system 184 is conducted by a surgeon to determine the holistic view with overlaid features and decides where to position the probe, perform laser treatments, acquire additional OCT volume images, and interact with the feature detection overlay system to highlight various features as needed.

The Positioning subsystem 186 includes the examination system that examines the combined holistic surgical view, with features highlighted, and positions the smart laser probe either manually or robotically. In one embodiment, the examination system may be an operator or surgeon, or a robotic system. In one embodiment, the x,y,z location of the smart laser probe within the surgical field is constantly tracked so that the system is aware and can record the probe's position within the surgical field and integrates new OCT image data into the combined holistic surgical view. Once in position, the examination system decides if he/she wants to perform a laser treatment or acquire a new OCT volume image to be integrated into the holistic view. The Surgeon Initiated Laser Treatment 188 determines if the surgeon decides to perform a laser treatment, the probe both delivers the laser energy and acquires OCT images simultaneously. The OCT images are processed real-time and used to control laser dosimetry.

In some embodiments, the imaging beam can has shared optical path as the laser system. The imaging system can also sue the same light source as the laser if required. The image guided system may integrate subsystems as described above to modules controlled by computer related systems. Calibration and controlled surgical procedures may be performed under the module control and implementation.

Conditions/Applications

The image guided system may be applied for a variety of medical applications and medical conditions, treatments, surgical procedures, and diagnosis. The medical applications elucidate on the effectiveness of the image guidance in laser-tissue modification and material deposition.

In one embodiment, the image guided system treats a myocardial infarct, which generally comprises the first step of tomographic imaging (OCT) collects and visualizes the epicedium to guide the physician. The image classification step helps in finding delicate blood vessels, ischemic tissue and the sites for microwell incision. The surgical objective drives the device to laser-cut microwells into the epicardium while avoiding unwanted damage to the vascular sites and, the tomographic imaging informs the injector to deposit angiogenic chemokines to penetrate the infarct at the wells. This site can be finally sealed with the tomographic image guidance controlling the co-aligned tissue modifying laser. Another application is for Chronic Total Inclusions (CTOs) where OCT is used to guide a cutting “wire” and insure that the vessel is not punctured by the cutting wire.

In another embodiment, the image-guided system treats cancer, where the image-guided system can provide highly localized chemotherapeutic or radiological-seed treatment to cancer margins as tumor tissue is imaged and classified in vivo, and then treated with the same image-guided system. The trend in tumor resection surgery has taken a turn to where maximum ‘normal tissue’ retention is the surgical objective. Hence, the image-guided system reaches the margins based on pathology results to the best of surgeon's abilities and drop patches/drugs/radioactive pellets in the location of resection to act as chemotherapeutic/radio therapeutic drugs to treat the patient. The image-guided system coupled to the tumor resection surgery process, allows for depth image guidance steps to decide on specific regions to insert/inject these materials. Also, the image-guided system with the tomographic image guidance helps decide on specific regions to use the co-aligned laser to cut tissue and inject these materials, and seal them with another step of laser modification of the surface to act a sealant to these drugs. Additionally, a tomographic imaging guided step of chromophore-assisted laser inactivation or a photo chemical modification can be performed to better eject/inject the drugs/material into these tumor resection locations to treat whatever is left of the cancer.

In another embodiment, the image guided system can treat damaged articular cartilage, where tomographic imaging reveals regions of damaged cartilage tissue, the image guided system then deposits autologous stem cells into microwells to improve their differentiation into chondrocytes and adherence to host cartilage. In one embodiment, in order to reduce the amount of cells needed for operating at a particular site an injection protocol is proposed where the cells are transported to the location by multiplexing cell laden hydro-gel with normal hydro-gel or other cell compatible gels to make sure there is no cross-contamination. After cell-laden gel contact and interaction is complete, a secondary conduit in the image guided system transfers the solvent location in order to seal as per need. In one embodiment, an additional OCT guided laser pass is made to induce/excite the cells for growth.

In one embodiment, the image guided system and device is integrated into a robotic surgical system (e.g. Intuitive Surgical daVinci) through an accessory port or one of its robotic arms. Several candidate tissue deposition sites can be quickly made during surgery, with real time imaging feedback provided for each sampled region following the work flow mentioned in FIG. 1a. The image guided system can be broadly used in a wide variety of surgical interventions for which real time characterization of the cell/drug/material deposition sites are needed.

The image guided system may be integrated to a biologic-injection system. The image guided system can be used to image real time and guide the process of cutting a small volume of tissue out and injecting cell/biologic/therapeutic material into it, with in vivo evaluation of tissues using OCT. OCT has been used in a group of surgical techniques which have been increasingly applying automated cutting procedures but material injection in tandem with co-aligned laser modification in tissue is a novel addition of this invention.

The image guided system employs the delivery of patient-specific/patient-controlled therapeutics/biologics at the sites of the laser-modified tissues (e.g. cutting, coagulation) while OCT guides the process of laser-modification without damage to adjacent structures. This approach can also deliver biocompatible solvents, directly to the tissue guided by tomographic imaging to rapidly close up wounds along with cells, biologics and therapeutics.

The image guided system solves problems in diagnosis and management of disease in the clinical setting. The image guided system has the potential to guide surgical resection in order to improve outcome for cancer, infarction, osteoarthritis, and other diseases that can benefit from micro-precise treatments. Angiogenesis in circulation starved tissue, like myocardial infarctions, can be directed with cytokines deposited into precisely patterned tissue wells. Cancer margins of tumor, which have been removed, can be imaged at the site and treated with radioactive seeds or chemotherapeutics. Micro-incisions can be made in cartilage to inject stem cells that will proliferate and differentiate with greater efficacy than those deposited in traditionally bored holes.

Most surgical interventions involving cell/cell laden material/drug injection rely on surgeon's expertise in finding the locations for delivery. For example, in the case of osteoarthritis the surgeon locates a few places of cartilage injection based on their expertise, but they do not have any tomographic imaging information on the diseased/non-diseased cartilage classification. With OCT, this can effectively be considered while doing imaging. With the image-guided system mentioned, a co-aligned tissue modifying laser and co-aligned injection mechanism, specific target spots can be localized or analyzed through the OCT and patient specific stem cell-laden hydrogel can be injected in the sites of micro-well creation. The image guided system and method, performed in vivo, can largely limit functional tissue damage. The image guided system provides micron level resolution of tissue which has been shown to be diagnostic of disease (e.g. cartilage disease, etc.). This method of microwell creation to promote cell viability and growth has been studied with promising results for over the past few years. The evaluation is commonly performed in scaffolds, but no depth-image guided in vivo method of injection has been reported. The results of the intra-operative evaluation of the cartilage can be communicated to the surgeon and consequently OCT can guide the surgeon's subsequent actions. Microwells allow cells to penetrate into the tissue and provide conditions suitable for cell growth, specifically the effective diffusion of materials and a surface which promotes cell adhesion. A desirable rate of diffusion of nutrients and oxygen is achievable within the micron-scale volume provided by a microwell. Cell adhesion is affected by the microtopography of a surface; specifically, cells adhere better to a smooth surface. Because the Thulium laser (or ultra-short lasers such as a Yterbium fiber laser) is capable of creating smooth cuts at the micron scale, laser-induced microwells provide an environment that both eases nutrient diffusion and encourages cell adhesion.

The image guided system may apply a comprehensive stem cell injection and drug delivery that has enormous potential for clinical use.

The image guided system and device can be used for identifying specific sites of pain in endometriosis patients for effective lesion removal and specific therapeutic drug delivery post-surgery can help largely limit the pain felt by the patient. For example, endometrial lesions can be located on delicate structures including ovaries or fallopian tubes and must be removed without damaging these underlying structures. At some sites, micro wells can be made to deposit these drugs to treat the patient effectively. Melanoma that has spread to soft tissue between wrist and shoulder can be treated with inter-tumor injections effectively by the image guidance provided by the device.

Image Guided Systems

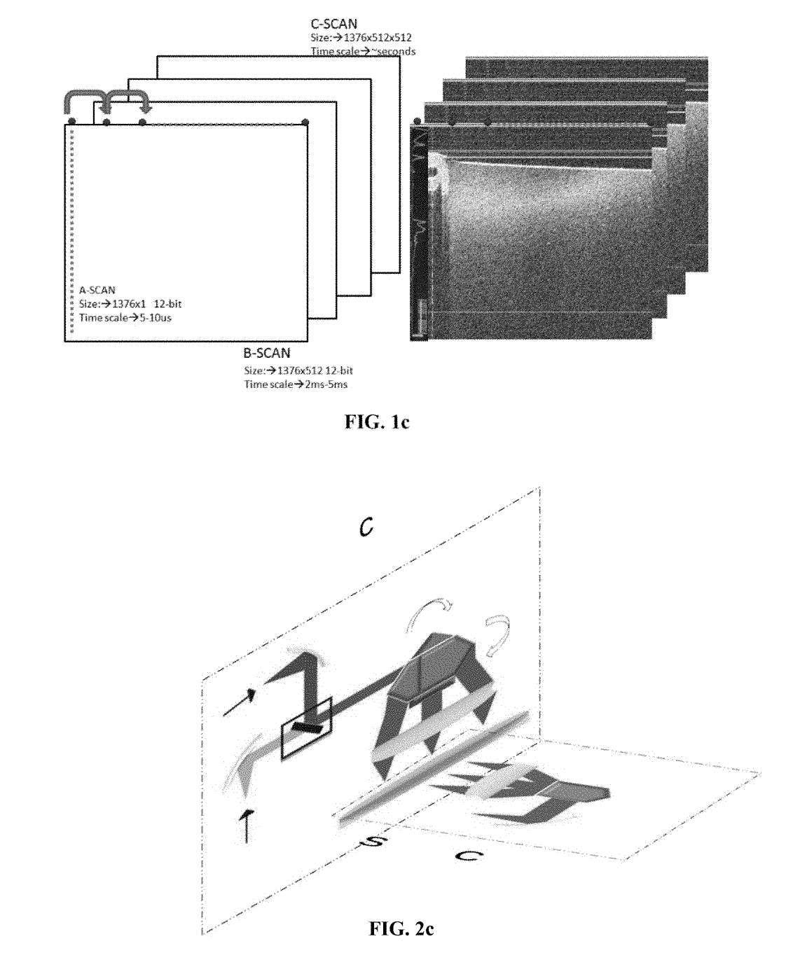

One embodiment of the image guided system is shown in FIG. 2a, which is an Image-guided laser 200 for cutting/cell Implantation. The Image-guided laser system 200 comprises an OCT imaging system 210, a laser system 230, and a Biomaterial/Cell deposition system 250. The Biomaterial/Cell deposition system 250 may or may not be incorporated into the image guided laser 200 system if cutting or cell implantation is not necessary. The OCT Imaging system 210 includes an OCT source 212. In one embodiment, the OCT source 212 is a swept-source mode-locked laser source centered at about 1310 nm±70 nm, with a fast scan-rate of about 100 kHz. The output of the laser enters a Mach-Zehnder interferometer setup, including a sample arm 214 and a reference arm 216, where the sample arm 214 is optically coupled to a circulator 224 and the reference arm 216 is optically coupled to a circulator 226. In one embodiment, the sample arm 214 and the reference arm 216 are path length and dispersion matched. Backscattered light from the reference and the sample arm interfere to form a fringe, which is detected using balance detectors 220 (BD). In one embodiment, the bandwidth of the source 212 is about 130 nm with a long coherence length of about 20 mm. In one embodiment, the lateral resolution obtained from the system is about 10 μm, and the axial resolution is about 7.5 μm in air. The sample arm 214 includes an Angled Physical Contact (APC) connector 228 to fiber deliver an OCT beam 218. The OCT beam 218 is reflected off a reflective collimator 222 to be operably coupled with the laser system 230.

In one embodiment, the laser system 230 is a nanosecond pulsed fiber laser system used for cutting tissue. In one embodiment, the average power of the laser is a maximum of about 15 W corresponding to a pulse energy of about 500 μJ per pulse, a pulse duration of about 100 ns and a repetition rate of about 30 kHz. The light the laser system is fiber delivered from an Angled Physical Contact (APC) connector 236 to and collimated using a reflective collimator 232 (RC08 Thorlabs Inc.) and directed onto a di-chroic mirror 234 (DM) which co-aligns a cutting laser beam 238 with the OCT beam 218 to produce a combined Laser/OCT beam 240. The combined Laser/OCT beams 240 are redirected through at least two galvanometer mirrors 242 onto a tele-centric aspheric ZnSe lens 244 (LSM, ISP Optics AR812-ASPH-ZC-25-25). In one embodiment, the laser beam focuses to an about 30 μm spot size, corresponding to a fluence of about 60 J/cm², where higher than the threshold of ablation caused by thermal confinement. The midpoint between the two scanning galvanometers 242 is positioned in the back focal plane of the aspheric ZnSe lens 244 to form a telecentric scanning system.

In one embodiment, the Biomaterial/Cell deposition system 250 deposits cells onto the modified tissue or phantom surface with an applicator tip 252. In one embodiment, the Biomaterial/Cell deposition system is a syringe applicator. In one embodiment, the syringe applicator is composed of two syringes 254, 256, one syringe 254 loaded with the cell-seeded-polymer and the other syringe 256 with a cross-linking agent, a mixing head to combine the two materials, and a syringe needle to direct deposition of the mixed hydrogel. The dual syringe is placed between about 250 μm to about 1 mm to the focal plane of the cutting of the thulium cutting laser to inject at the site of the micro-well cut.

The image guided system and method involves using the OCT system 210 to first position the applicator tip 252 to the micro-well and then adjusting location of subsequent images to capture the hydrogel deposition. The image guided system and method includes an applicator-optical mount created in order to have rapid, reproducible imaging of cell deposition that will include the tip. The applicator-optical mount may be designed using CAD and subsequently 3D printed in order to interface directly with the optical table mount and the syringe needle. The syringe needle will be oriented so that the end will appear in the background, perpendicular to the fast-axis, and will end at the focal plane. This way, modified tissue surfaces can be taken directly up to the needle and deposition can be imaged without adjustment.

Handheld Interface

One embodiment of the image guided system is shown in FIG. 2b, which is a hand held interface 300. The handheld interface 300 streams OCT images to the user and a processing element while simultaneously cutting the tissue with the laser system. The handheld interface includes a single axis galvanometer 310 (GVS002 Thor Labs Inc.) that is co-aligned and collimated with a laser beam 320 and an OCT beam 322 (collimated via RC04 reflective collimators 312 and RC08 reflective collimator 314 and co-aligned via a dichroic mirror (DM) 316. The OCT beam path begins at the RC04 collimator 312 mounted on an XY micrometer (CXY, Thor Labs Inc.) that is used to adjust and co-align OCT and laser beams 324; the co-aligned beams then reflect off a gold mirror 326 (shown in the FIG. 2b after the dichroic mirror) and then onto the galvanometer 310. The galvanometer 310 was positioned at the front focal plane of an aspheric ZnSe lens 328 (AR112-ZC-XWL-25-25, ISP Optics).

### Alternative Embodiments

The image guided system and methods may include additional cell viability and contamination avoidance. The image guide system and methods may include: 1) a rapid flush of cleaning solvent in order to “wash” any remaining compounds from previously cell/material injection procedure; 2) disposable probe tips included in the cell deposition method.

The image guided system may be operably coupled with a tissue sampling element. The tissue sampling element may be a mass spectrometer to guide the histology of the tissue or sample as to determine disease, pathology, and condition of the tissue or sample.

FIG. 2c is a schematic of a forward looking cutting laser coupled with a side cutting laser.

### EXAMPLES

Hereinafter, the present invention is more specifically described by way of examples; however, the present invention is by no means limited thereto, and various applications are possible without departing from the technical idea of the present invention.

### EXAMPLE

### Image-Guided System for Precision Implantation of Cells in Cartilage

Introduction

Osteoarthritis (OA) is a degenerative joint disease that is the most chronic form of arthritis and impacts nearly 27 million people in the United States. People suffering from OA experience chronic pain, impaired mobility, rapid fatigue and increased risk of injury. Due to the severity and prevalence of OA, current research focuses on articular cartilage repair and regeneration. A potential long-term solution to treat OA is stem cell-based replacement therapy that allows implanted cells to differentiate into chondrocytes thereby promoting cartilage regeneration. However, the development of an effective stem cell therapy for OA is limited by three problems that result in low retention and survivability of stem cells in vivo. First, no in vivo imaging method is utilized to identify candidate regions in the articular cartilage for stem cell implantation. Second, the relatively large-diameter mechanical tools that are currently utilized to create receiving wells in articular cartilage are too coarse and do not allow the implanted stem cells to communicate with surrounding articular cartilage. Third, stem cells must be delivered in a medium that enhances their survivability and promotes differentiation into chondrocytes.

These problems can be solved fivefold: 1) compared to conventional arthroscopy, OCT provides three-dimensional imaging allowing volume visualization of articular cartilage. OCT imaging of articular cartilage correlates with arthroscopy and T2 MRI. OCT can generate contrast between normal and diseased cartilage; thus, stem cell implantation sites can be identified by using this contrast. 2) A co-aligned fiber laser (e.g., Tm, Yb, or similar) can create small-sized receiving wells for stem cell implantation and the size of these wells can be verified with OCT; partial repair of laser irradiated articular cartilage by controllable laser-assisted pore formation in the cartilage matrix. Pore creation in cartilage promotes increased mass transfer of nutrients and signal molecules that enhance triggering processes required for stem cell differentiation; 3) Stem cells laden iHA hydrogel can be injected into receiving wells through the image-guidance from the OCT; 4) modification of the cartilage regions surrounding the implantation sites can be done via for example a nanosecond thulium (Tm) or similar fiber laser to enhance transport nutrients and signaling molecules; 5) OCT can monitor response of the cartilage at selected time points following stem cell implantation. Microwells allow cells penetration into the tissue and provide conditions suitable for cell growth, specifically the effective diffusion of materials and a surface which promotes cell adhesion. A desirable rate of diffusion of nutrients and oxygen is achievable within the micron-scale volume provided by a microwell. Cell adhesion is affected by the microtopography of a surface; specifically, cells adhere better to a smooth surface. Because a fiber laser (e.g., Tm or similar) is capable of creating smooth cuts at the micron scale, laser-induced microwells provide an environment that both eases nutrient diffusion and encourages cell adhesion.

In this example, the image guided system combines advanced laser imaging and tissue modification with stem cell implantation for cartilage regeneration and treatment of osteoarthritis for stem cell impregnation and laser-assisted pore formation and regeneration. As the growth of hyaline cartilage can be accomplished for a specific range of cartilage modification, optical imaging (OCT) of laser-induced thermo-mechanical strain and structural alterations is vital for efficacy and safety of OA laser treatment. This example demonstrates OCT's capability to image real-time ablation of a tissue analogue and the deposition of hydrogel into a surface modified phantom. An applicator device delivers the seeded iHA to the modified site. Viability tests will be performed to ensure the applicator is not inducing apoptosis in the hMSCs by either cytotoxic or mechanical stresses during delivery from syringe to micro-wells.

Therefore, this example shows the image guided system for laser based treatment of OA, which combines laser cartilage repair, stem cell implantation and OCT.

Methods

In the first part of this section, the image guided system is used for imaging/cutting/deposition. In the second part of the section, the experiment design was carried out to ascertain the versatility of the image guided system.

FIG. 2A shows the image-guided system used for cutting/cell Implantation. As described previously, the image guided system comprises three major subsystems: (1) OCT imaging system (2) Nanosecond pulsed fiber laser system (3) Biomaterial/Cell deposition system.

Experiment Design

A literature survey of optical properties of cartilage shows that 80% water-gelatin phantoms match the absorption properties of cartilage. The laser-tissue interaction of the cutting beam on the phantom closely resembles the interaction with cartilage, given the same absorption coefficients at 1.94 μm wavelength. The pulsed laser fluence at the focal plane is 60 J/cm². Precision incisions are made using the image guided cutting laser procedure to create these microwells for deposition.

Due to the relatively high expense of producing hMSCs, a cell analogue may be an option to perform viability testing. The current candidates are 3T3 mouse fibroblasts, which have been selected based on their low cost and convenience to maintain, as well as their phenotypic similarities to chondrocytes as connective tissue cells. The fibroblasts will be grown as a continuous cell line and divided when cells are required for testing. These cells will then be detached, resuspended and seeded into iHA for deposition through the syringe applicator. Seeded hydrogel will be deposited onto laser-modified cartilage tissue explants or gelatin phantoms.

Calcein AM is the current candidate for the viability stain, for its extensively documented use, ability to penetrate hydrogels, and reliable fluorescent signal. Once the hydrogel has set, the filled microwells can be sectioned and subsequently hydrated using a Calcein AM solution. Samples will be taken from seeded hydrogel, which has been deposited from the applicator into the microwells and from seeded hydrogel applied directly to microwells. All samples will then be examined under a fluorescent microscope and their intensities compared.

Results

Image Guidance for precision laser cutting in tissue phantoms (Demonstration of the versatility of the cutting process)

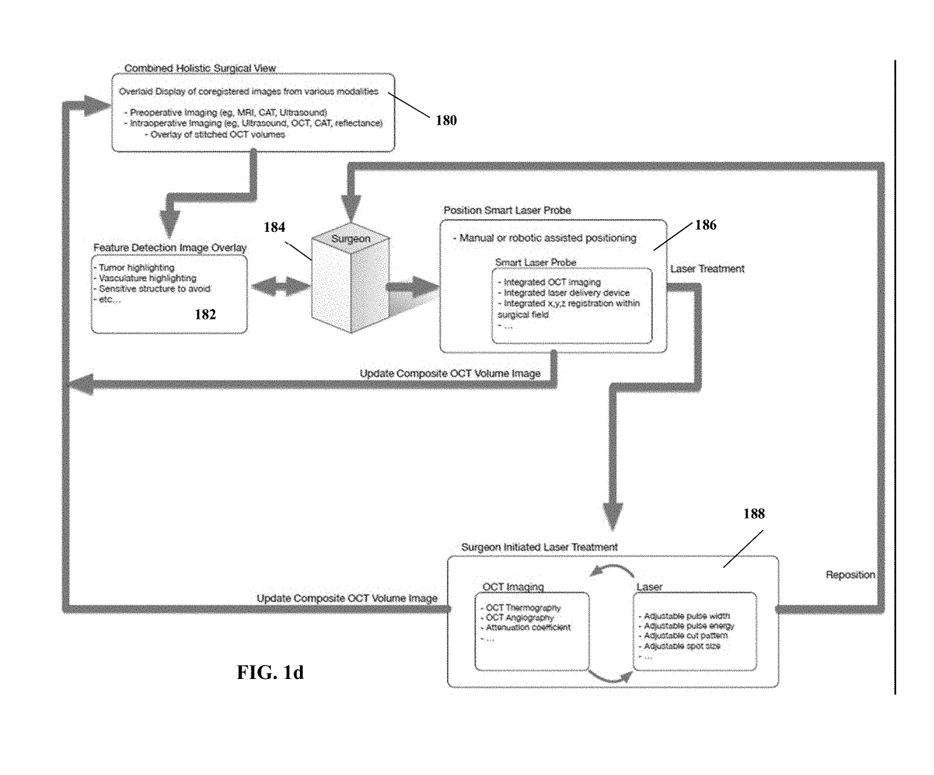

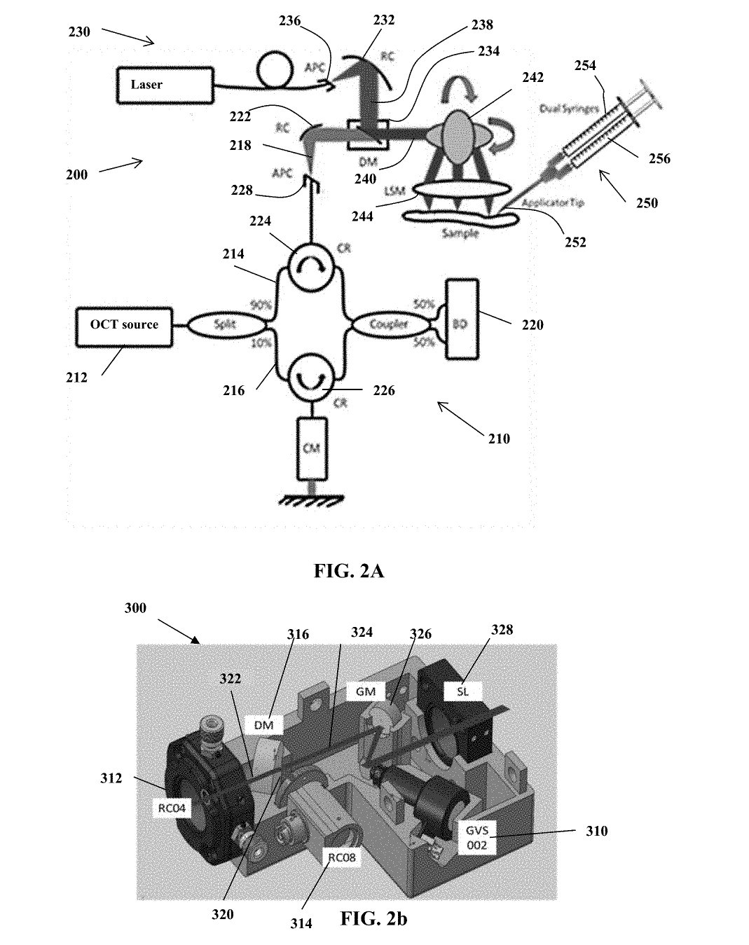

The OCT image-guidance informs a fiber laser for cutting/removal of targeted tissue structures. Using the 80% water tissue phantoms, surgical incision is possible with the laser, where 1 mm wide, 400 μm deep cuts are made executed OCT to guide the cutting procedure. FIGS. 3, 4 shows the cutting process carried out in enface and cross-section images along with the time-lapse images of the cutting process observed under the OCT. The white arrow in FIG. 4 shows the tissue material being blown off the top surface of the tissue.

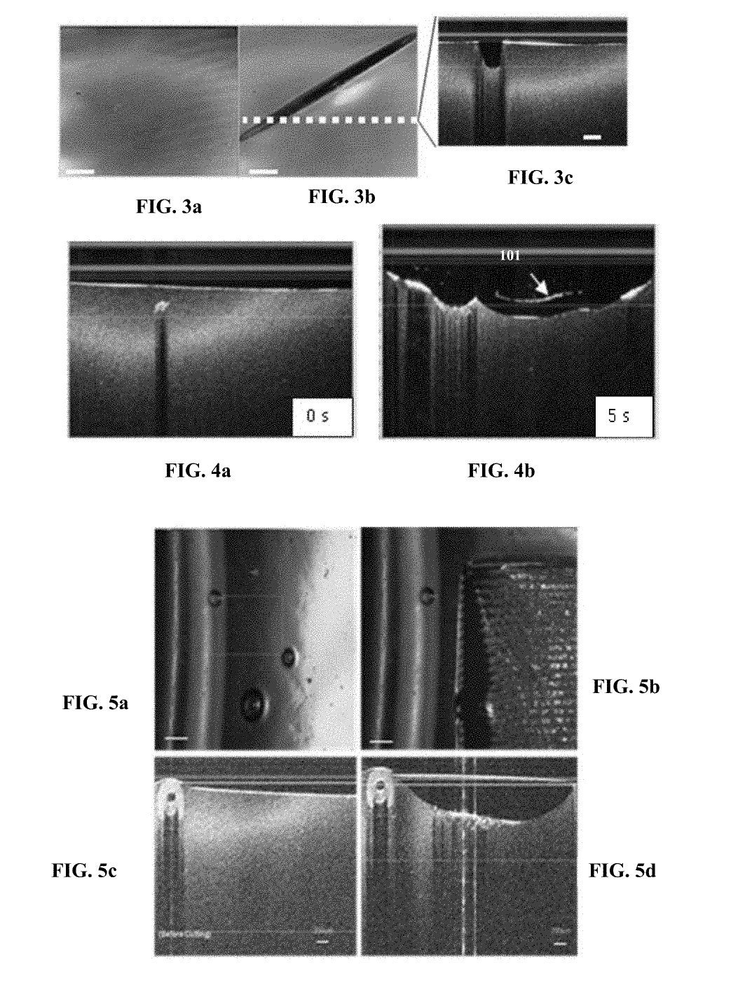

FIGS. 5a-5d is an automated OCT Image guidance to control the fiber laser to cut around structures OCT versatility showcased in the creation of cutting sites while automatically avoiding structures (in this case the micro-vessel on the surface); where FIGS. 5a, 5b are enface images of the phantom before and after the formation cutting with the laser. And FIGS. 5c, 5d are the cross-section image of the phantom. Scale bars are 200 μm

Image Guidance for Precision Material Deposition in Tissue Phantoms:

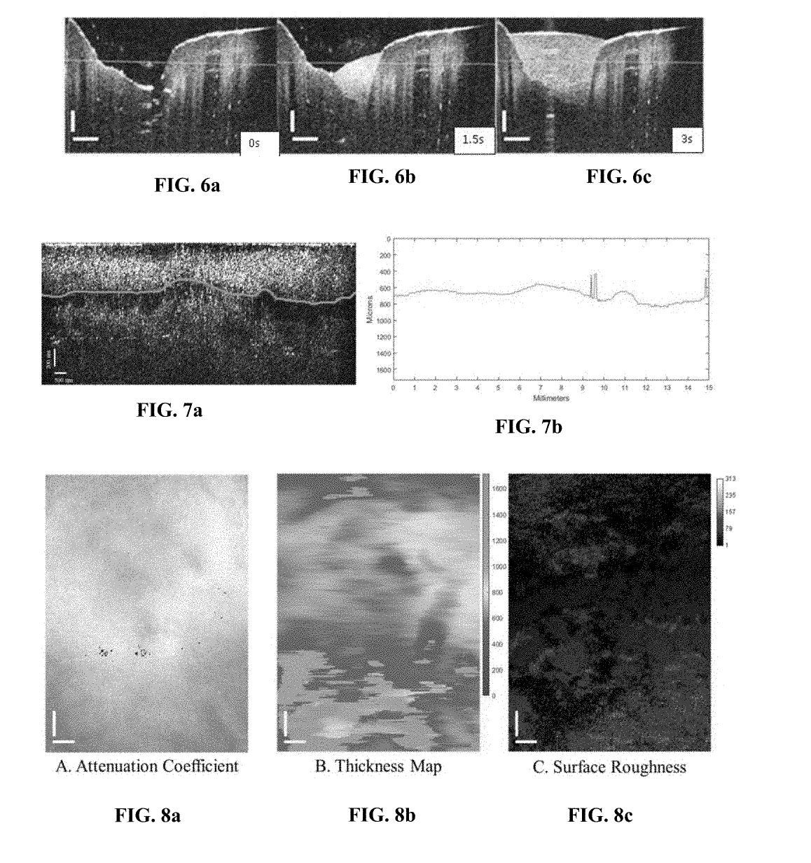

The injecting device is imaged in the space of the incision, and OCT image-guidance informs the user about the flow of the deposition into the incision. This feature was showcased using milk:water:gelatin (40:40:20) ratio solution and the heated solution was permitted to flow into the incision and solidify-all the while imaged by the OCT real-time. FIG. 6 shows the real-time time lapse images of the deposition process.

Conclusion

In this example, the image guided system combines advanced laser imaging and laser tissue modification with stem cell implantation for cartilage regeneration and treatment of osteoarthritis for stem cell impregnation and laser-assisted pore formation and regeneration: an equivalent system has not been described in literature about such an image guided system for cell deposition. As the growth of hyaline cartilage can be accomplished for a specific range of cartilage modification, optical imaging (OCT) of laser-induced thermo-mechanical strain and structural alterations is vital for efficacy and safety of OA laser treatment. Therefore, the image guided system is shown for laser based treatment of OA, which combines image-guided laser cartilage repair with stem cell implantation.

### EXAMPLE

### Cartilage Determination Parameters

Using OCT, it is possible to observe and analyze parameters that are linked to the cartilage condition. This work investigates the use of attenuation coefficient, thickness, and surface roughness as metrics to assess the health of articular cartilage of the knee. The image guided system can be used to locate areas of diseased cartilage and to designate them as sites for treatment.

Methods

Image processing and analysis of OCT scans were performed using ImageJ and MATLAB. To create a thickness map for a region of cartilage, the OCT images were first adjusted to account for the offset between the surface of the cartilage and the top of the image. A band-pass FFT filter was then applied to isolate the signal generated by the cartilage/bone boundary. MATLAB was used to find the number of pixels from the top of the image to this boundary in each a-scan. Each point was then assigned a thickness value corresponding to this distance. The resulting thickness values were mapped to their respective locations and displayed as an enface image.

The surface roughness of cartilage was measured using the ImageJ plugins Extended Depth of Field and SurfCharJ which performed a gradient analysis on OCT images.

Results

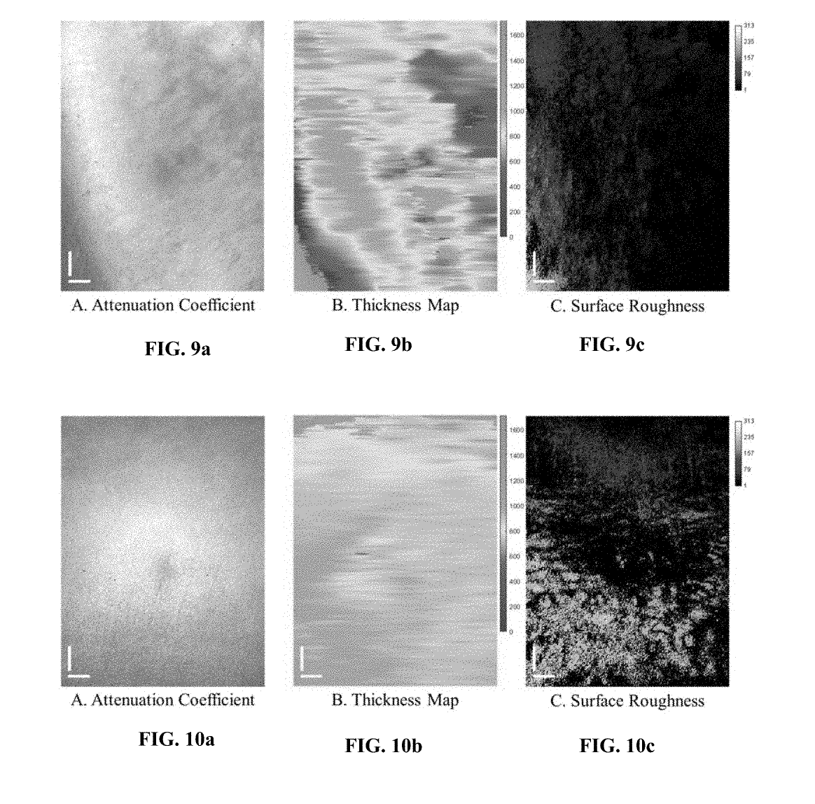

OCT imaging was performed on different regions of articular cartilage from a porcine knee. The images were then analyzed using the cartilage tissue metrics mentioned previously. In the thickness map, cartilage areas of greater thickness are displayed in green while areas of less thickness are displayed in red. The technique used for thickness mapping was tested by applying to individual b-scans. The resulting thickness values were then plotted and compared to a line tracing of the cartilage/bone boundary as shown in FIG. 7. For the surface roughness assessment, dark blue areas correspond to areas with fewer large polar angles in a gradient analysis and are thus smoother than bright yellow areas.

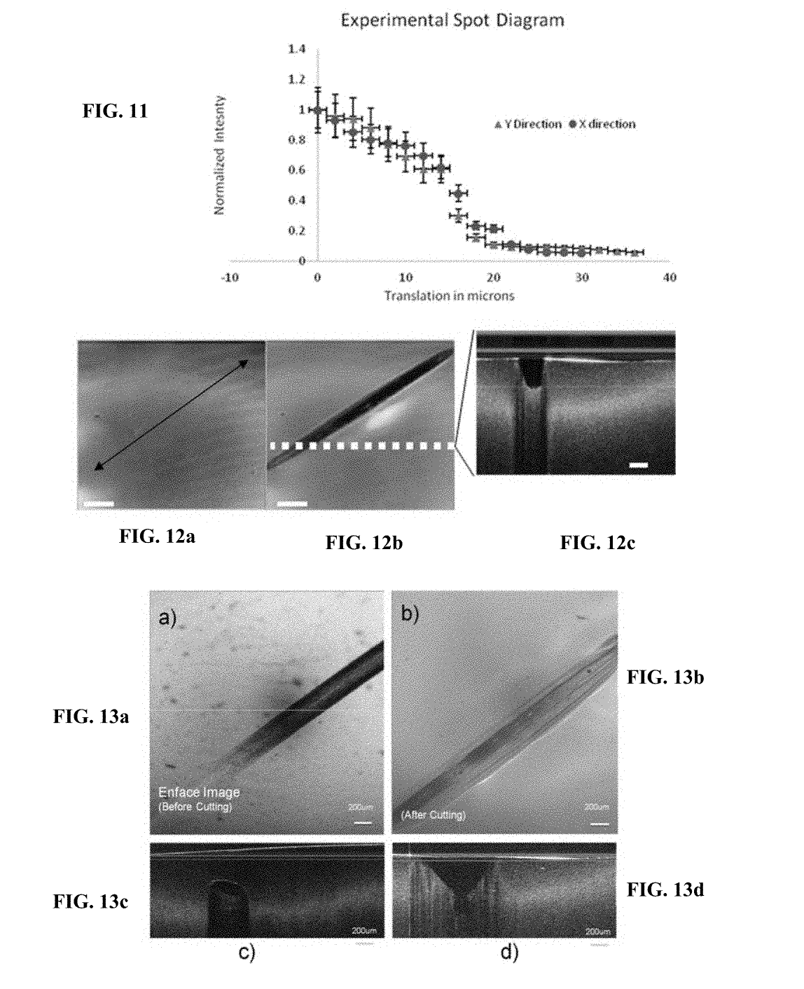

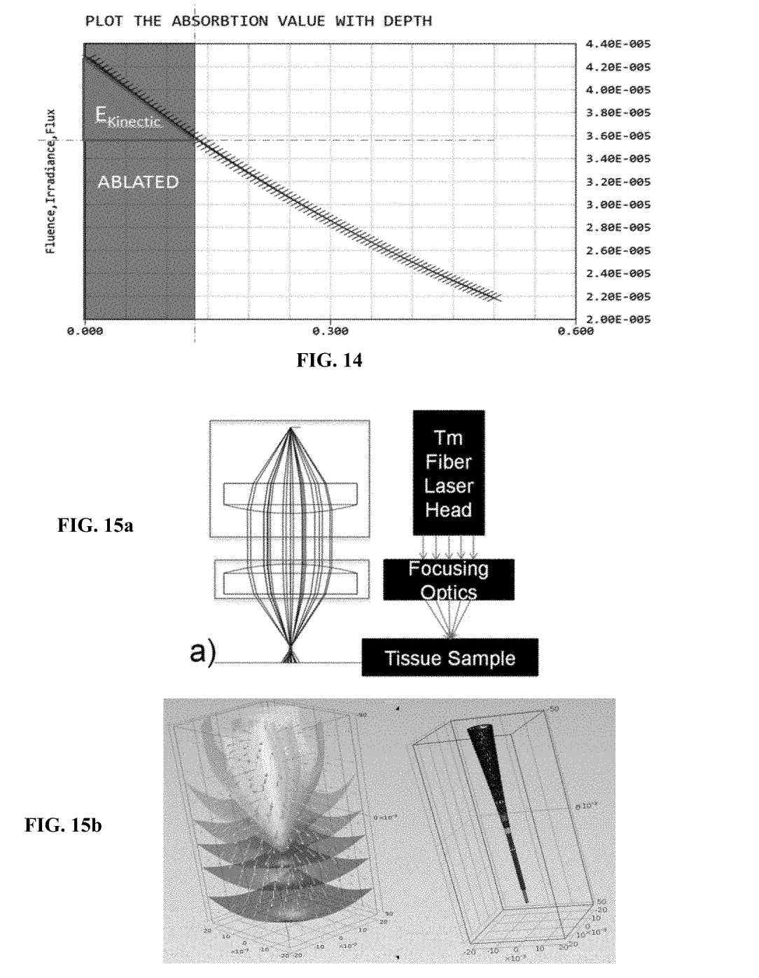

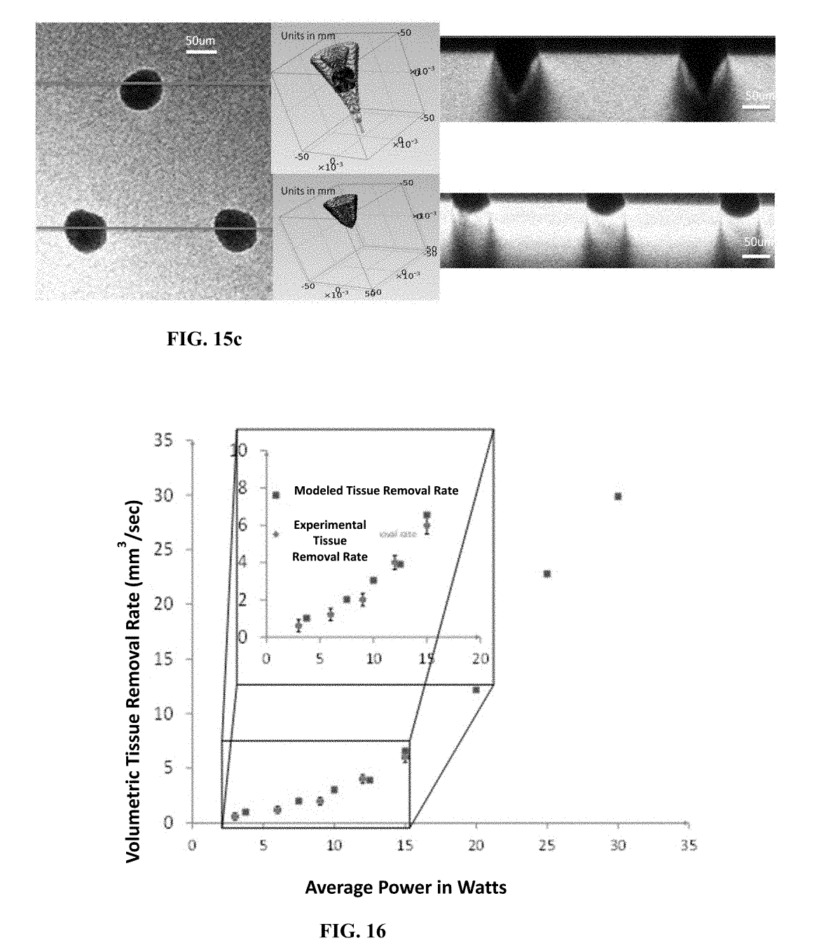

FIGS. 8, 9, and 10 show the attenuation coefficient, thickness map, and surface roughness of different regions of cartilage. This study shows that by using OCT, it is possible to assess cartilage based on metrics that are indicative of cartilage health. A possible future exploration could be conducted to see how this data correlates to histology. Furthermore, histology would provide insight on what values correspond to diseased and normal cartilage using these parameters. The significance of this study lies in having a method and set of parameters which could be feasibly used to examine cartilage and determine diseased areas as potential sites for treatment.

### EXAMPLE

### OCT Image Guidance for Surgery and Cancer

Modeling and Experiment Design

A literature survey of optical properties of tissues suggests that gelatin phantoms made of 70-80% water (weight/volume) match the absorption properties of most tissues. The laser-tissue interaction of a laser cutting beam with the phantom simulates the interaction with tissue, considering similar absorption coefficients at a 1.94 μm wavelength.

Optics in the laser path were simulated using a ray-optic simulation software (Zemax). The simulation was completed in a non-sequential mode to obtain the fluence/flux in a volume of the tissue sample. The ablated region of the sample was obtained by using the “blow-off model”^{15,16}. The obtained threshold value was related to the enthalpy of ablation (hₛ) given by Eq. 1.

$$\begin{matrix}{F}_{th} = \frac{{h}_{a}}{μ} & ( 1 ) \\ Ω ( τ ) = ln \{ \frac{C ( 0 )}{C ( τ )} \} = {\int }_{0}^{τ} A \times {e}^{[ \frac{- {E}_{a}}{R \times T ( t )} ]} d t & ( 2 ) \\ Damage ( \% ) = 100 \times ( 1 - {e}^{- Ω ( τ )} ) & ( 3 ) \\ R = V \times \frac{PRR}{{P}_{Avg}} ( \frac{{mm}^{3}}{{W}_{s}} ) & ( 4 )\end{matrix}$$

The ablated volume of voxels was removed from the 3D tissue object and exported to a SolidWorks file importable into a finite element modeling software (COMSOL). These voxels represented the portion of tissue that is “blown off” in response to pulsed laser irradiation. In the finite element model, an initial temperature map was generated using the absorbed energy flux. Computed flux from the simulation was exported into the finite element model. The resulting lateral and axial heat diffusion was simulated by solving the heat diffusion equation. The fractional damage (%) was calculated (equations 2 and 3) using an Arrhenius damage integral. The tissue removal rate was computed using equation 4, where V is the volume of the voxels (each pixel 0.4 μm×0.4 μm×1.2 μm) removed by that were above the ablation threshold. Here, PRR is the pulse repetition rate and P_{avg} is the average power of the laser in Watts.

Results

Image-Guide System:

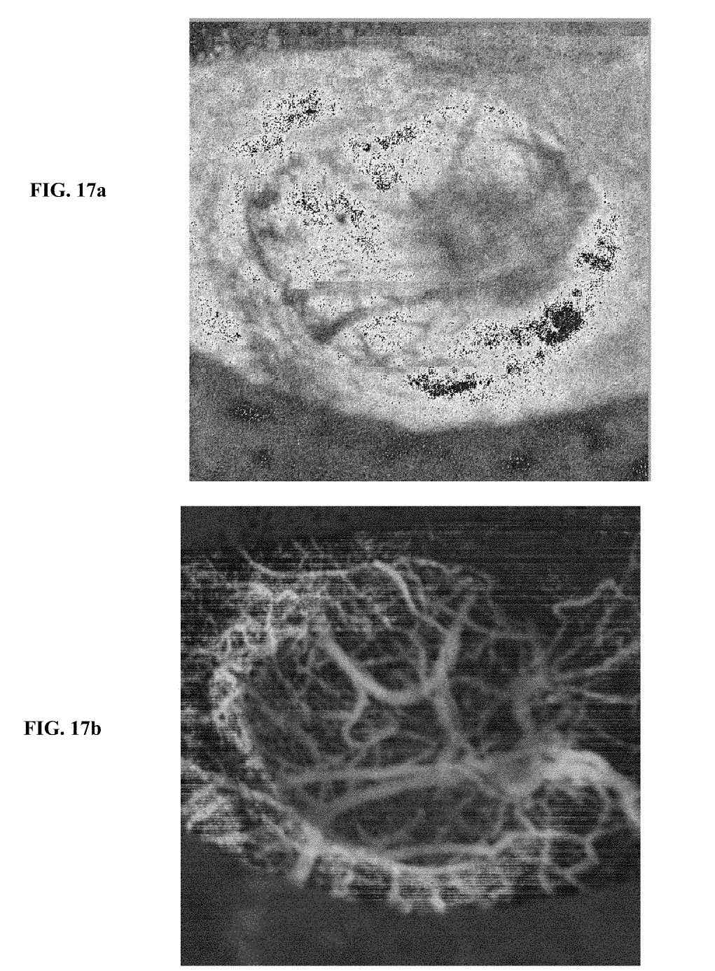

The first system characteristic verified related to the Image-Guide system was the spatial profile of the laser at the focal plane of the scanning lens. A custom in house-designed fast detection scheme using an InGaAS (G12182-003K, Hamamatsu) detector was used to record the intensity profile of the focused Tm-beam at the back focal plane of the 25 mm focal length scanning lens via use of precision mechanical stages (Aerotech) and placing the detector just behind a 2 micron diameter pin hole (P2S, Thor Labs Inc.). The x,y,z-stages were positioned carefully to obtain the optimal spot of the highest intensity and the spot size was estimated along one axis by translating the pinhole using a precision micrometer stage. The recorded lateral beam profile is shown in FIG. 11.

The focal spot's impact on the tissue was characterized using the OCT image to find the optimal Z-height in the OCT image to obtain the maximal tissue removal for cutting into the tissue. The airy disk spot size calculated from the Zemax simulation of the laser was about 20 um, which matched closely to the experimental beam profiling result.

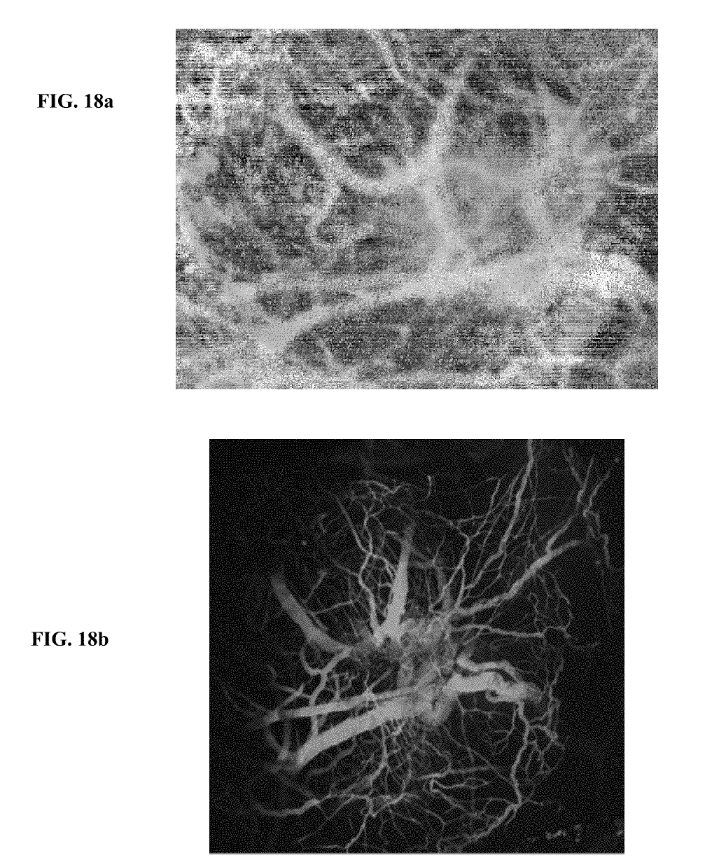

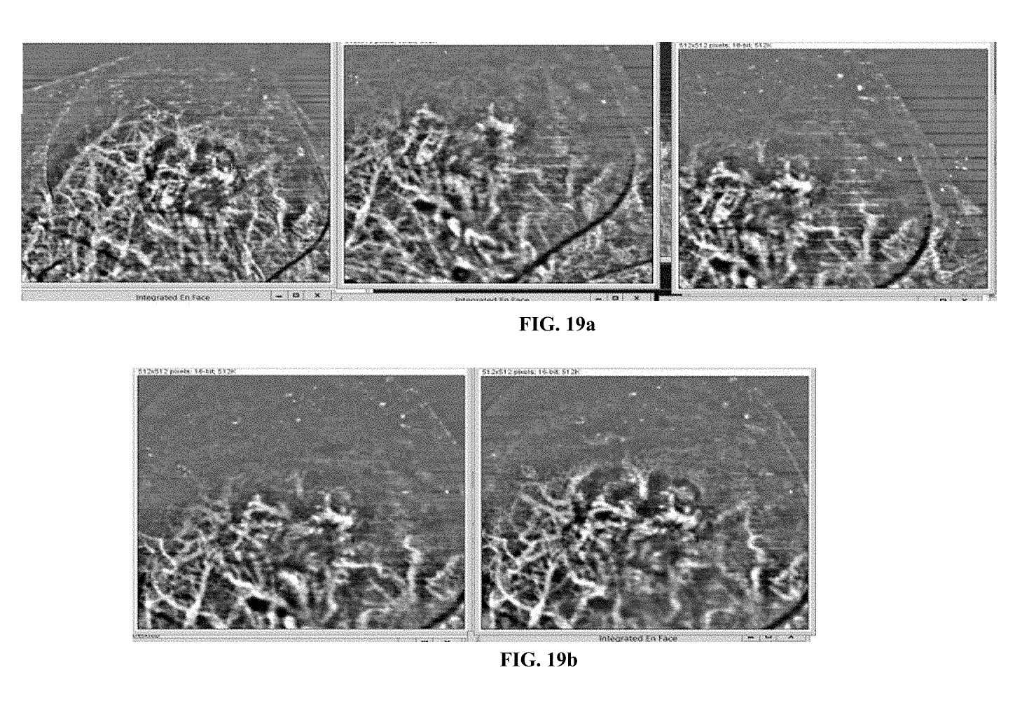

Image Guidance for precision laser cutting near sensitive physiologic structures (e.g., blood vessels) in tissue phantoms (Demonstration of the versatility of the cutting process): The OCT image-guidance system informs the laser for targeted cutting/removal of tissue structures. Using the 80% water tissue phantoms, a surgical incision was demonstrated (1 mm wide and 400 μm deep) created with the laser. FIGS. 12, 13 illustrate the cutting process with enface and cross-sectional images.

Image Guidance for Precision Laser Cutting Around Blood Vessel Phantoms (Demonstration of the Versatility of the Cutting Process)

OCT image guidance accuracy was demonstrated by performing a cut directly adjacent to a vessel demonstrating that material can be removed within a few microns away from a phantom vessel. The before and after images of this cutting process can be observed in FIGS. 5a-5d (FIGS. 5a, 5b as the enface view and FIGS. 5c-5d as cross-section images). FIGS. 5c-5d shows that the OCT guided cutting laser can be used to remove an entire section of material while still avoiding a vessel.

Handheld Device: Cutting Demonstration With Live B-Scans

The hand held interface was used to record real-time B-scan images (200 b-scans per second) for cutting into tissue phantoms. FIGS. 4a-4b highlights the images obtained in B-scan live mode along with the time-lapse images of the cutting process observed using OCT.

Laser Cutting: Modeling and Experimental Results:

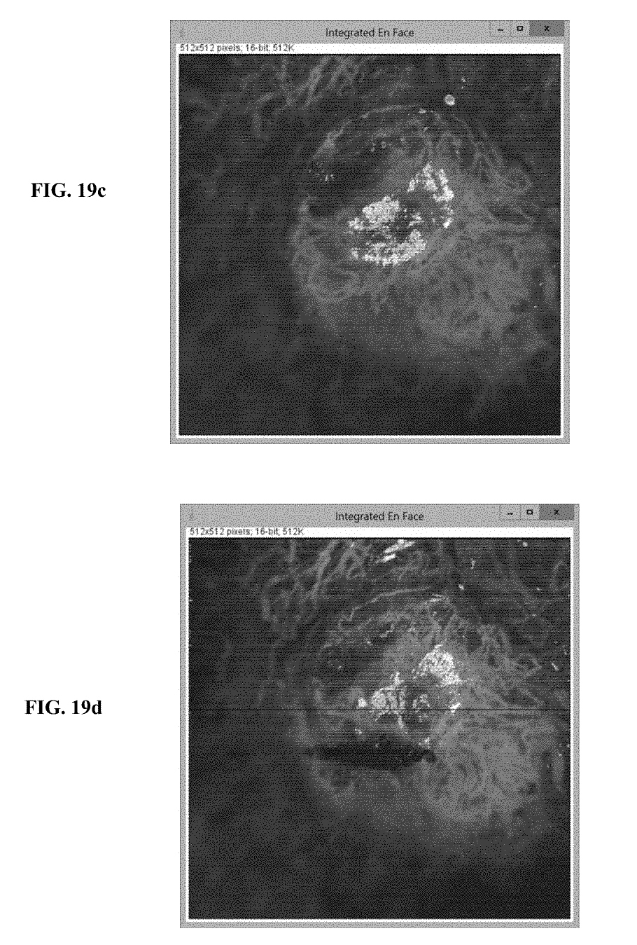

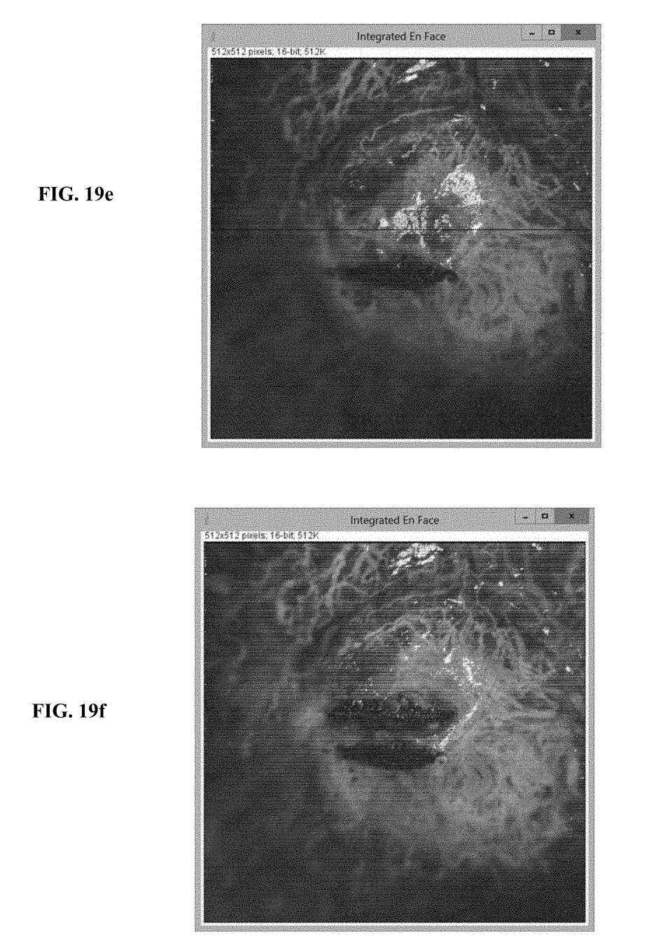

The simulated absorption (the computed flux) of a Tm-cutting model is shown in FIG. 14. The ablated volume of voxels was removed from the 3D tissue object and exported to a SolidWorks file importable into finite element software (COMSOL). The ablated volume from the optical simulation and finite element imports are shown in FIGS. 15a, 15b. The gelatin phantoms were used to simulate two different cases of the location of the sample with respect to the focal spot of the beam (100 μm and 200 μm respectively). The simulation results were compared to experiments as shown in FIG. 15c. These were obtained using a two-photon imaging technique with flourescein embedded in the gelatin.

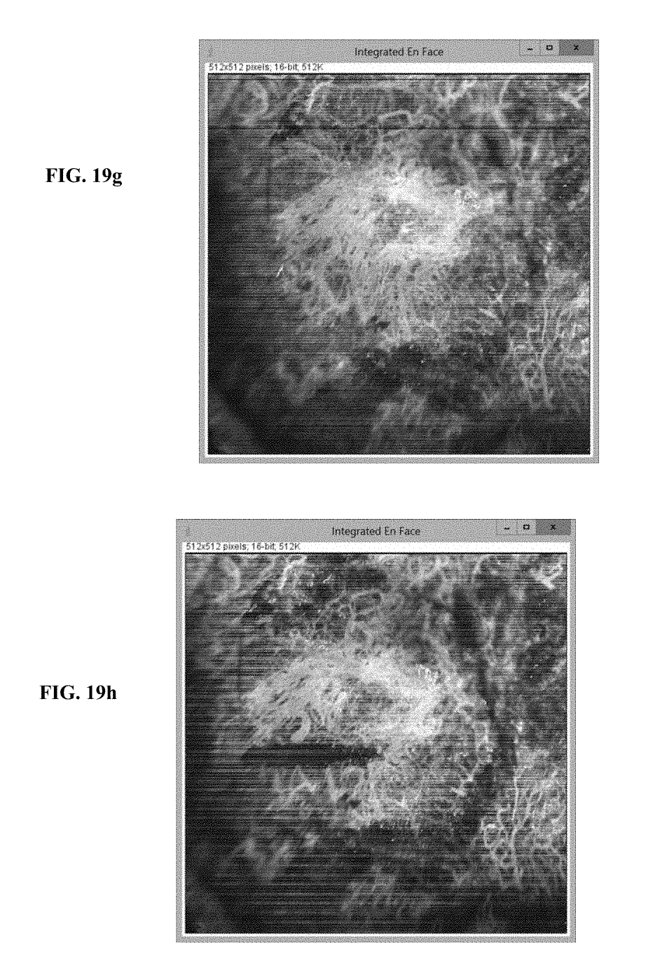

The tissue removal rate of the gelatin phantoms was obtained at different incident laser powers at a fixed PRR. The comparison of the modeled and the removed tissue rate obtained experimentally is as shown in FIG. 16. The OCT imaging provided a control signal to the laser's input trigger. From the OCT image, precise location for ON and OFF regions were sent to the laser for each B-scan and the trigger pulse controlled the location of cut on the tissue. OCT imaging feedback helped confirm these cutting sites and enabled calculation of removed tissue volumes using the total number of voxels removed from the volumetric images. The results were compared to predicted tissue removal volumes from the blow-off model and plotted in FIG. 16. The maximum incident laser power was limited to 15 W where as the model included values up to 30 W.

### Conclusion

During surgery, the lack of depth information, before cutting in tissue is detrimental and may lead to damage to critical blood vessels and delicate structures. Optical coherence tomography offers micron resolution (with millimeters of depth information) for imaging such critical structures and vessels. Combining this with a surgical laser, has potential application to precision tissue cutting.

The image guided system that combines optical coherence tomography (OCT) and laser tissue modification with a fiber laser [thulium (Tm)]. A modeling of the process was carried out using COMSOL and Zemax simulation tools. The simulation results of the cutting depth show good agreement to experimental cutting depths. The OCT image guided laser knife demonstrates the use of tomographic imaging to differentiate between types of tissues and can avoid damage to sensitive structures and still offer high speed micro-precision cutting at rates up to 5 mm³/sec. OCT imaging analysis to differentiate between cancerous and non-cancerous tissues. The mouse brain imaging example below indicates the ability to differentiate normal from tumor regions. Given the scalable potential of thulium lasers, use of a higher power cutting laser to provide faster tissue removal rate up to 100 mm³/sec is a second objective for this work. A study of pulse duration of the cutting laser with the amount of tissue removed and tissue damaged may better apply the image guided system under different settings to explore the possibilities of using the seed pulse shaping to achieve desired cuts and cutting-speeds.

### EXAMPLE

### Tumor Detection and Coagulation in Brain

Methods

Nude mice models with brain tumors were used for imaging with the device. The animals were anesthetized during imaging and were placed under the imaging system with a stereotactic mount. The craniotomy was located from the contrast generated in native OCT imaging. Then, blood flow contrast and attenuation contrast images were calculated from the native OCT images. The images were then compared to flow contrast images obtained from confocal imaging to ascertain the blood flow imaging contrast from the OCT. This part of the experiment was to showcase the tumor margin generation and blood flow contrast generation capabilities of the device. Alternatively, another mice experiment was carried out to demonstrate the coagulation capabilities of the device. The process starts again with locating the craniotomy and obtaining a blood flow contrast image. From the bloodflow contrast image, subsurface vessels are located. A zoomed-in cross-section imaging process was carried out to apply flow contrast at a location with blood vessels. Coagulation of these subsurface blood vessels was then carried out, encompassing the coagulation capability of the device.

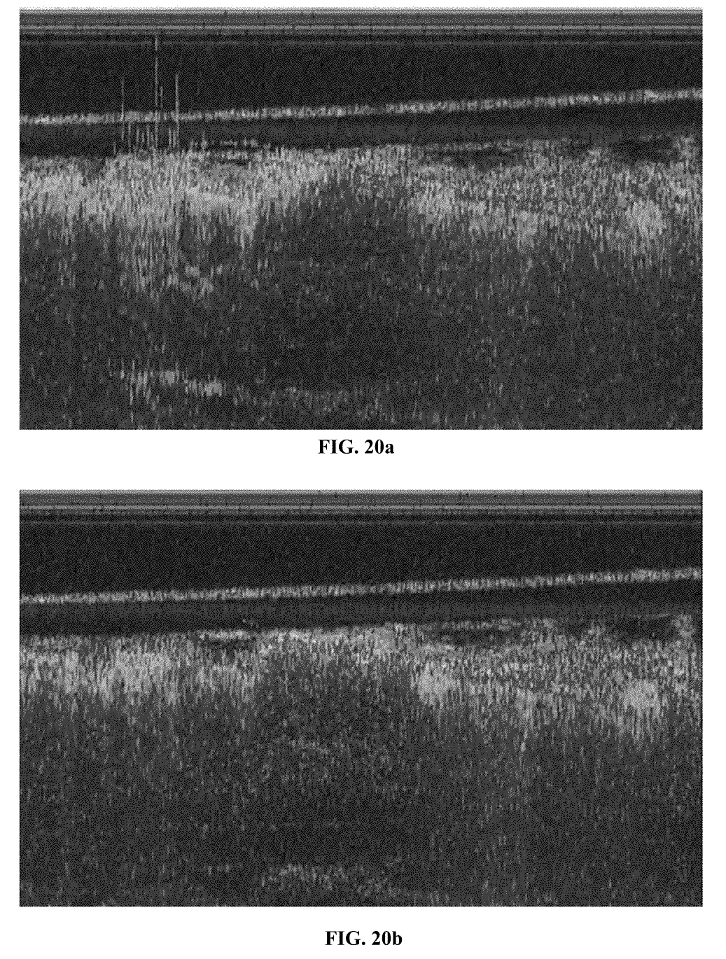

As shown in FIG. 17a, the dark regions in the attenuation coefficient image derived from the native OCT-image is indicative of the tumor locations. The blood flow OCT angiogram is shown in FIG. 17b.

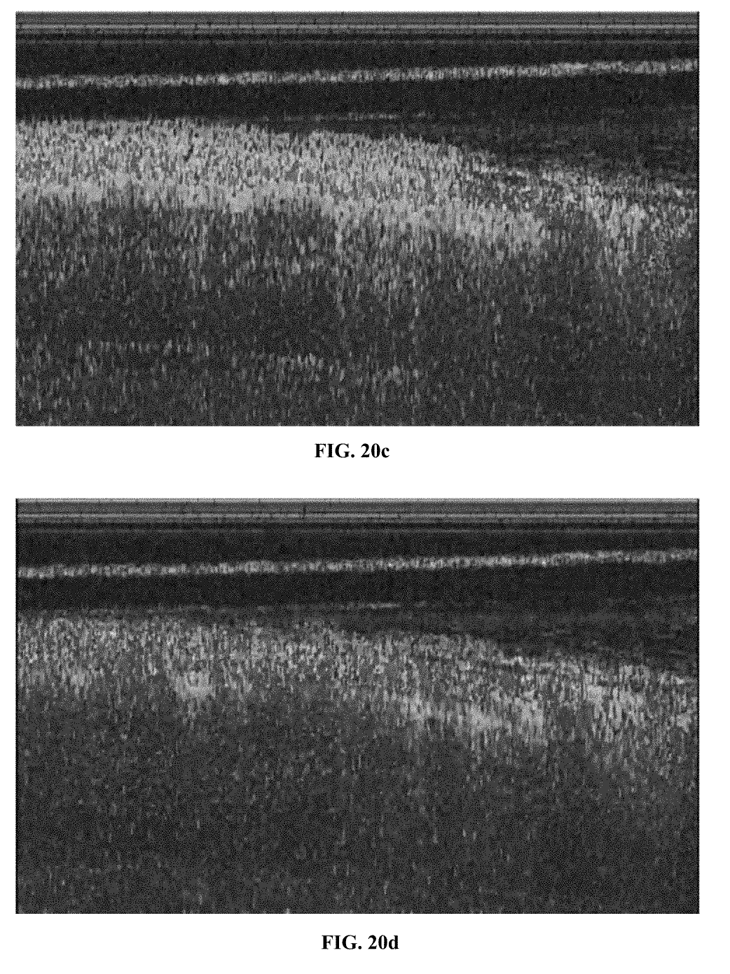

FIG. 18a shows the attenuation+flow overlay and FIG. 18b shows the fluorescence comparison recorded using the injectable contrast agent indocyanine green, where OCT can see the actual size of the tumor and is matched with the fluorescence image in terms of the blood flow.

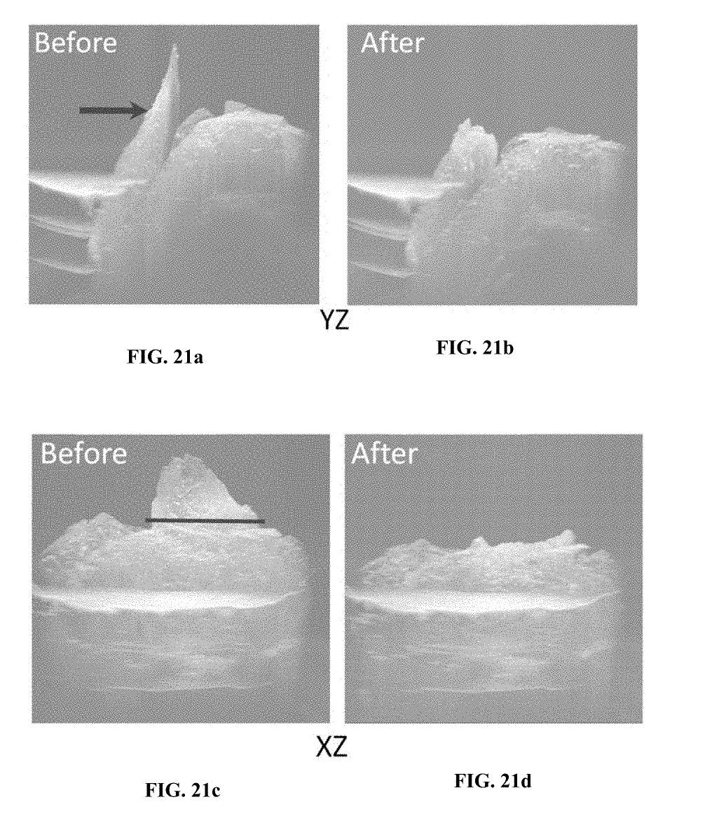

FIGS. 19a-19h demonstrates coagulation. Each of the sub-images are enface images of blood flow contrast calculated after each pass made by the coagulation process guided by the OCT imaging. There is a clear reduction in the number of blood vessels from left to the right in FIGS. 19a-19b, which include five passes of the surgical laser power at 0.5-1 W of a Tm laser. FIG. 19c is an enface OCT image before coagulation and FIG. 19d is an enface OCT image after 1 pass of laser irradiation showing coagulation. FIG. 19e is an enface OCT image after 1 pass of laser irradiation and FIG. 19f is an enface OCT image after 2 pass of laser irradiation showing coagulation. FIG. 19g is an enface OCT image before coagulation and FIG. 19h is an enface OCT image after 1 pass of laser irradiation showing coagulation.

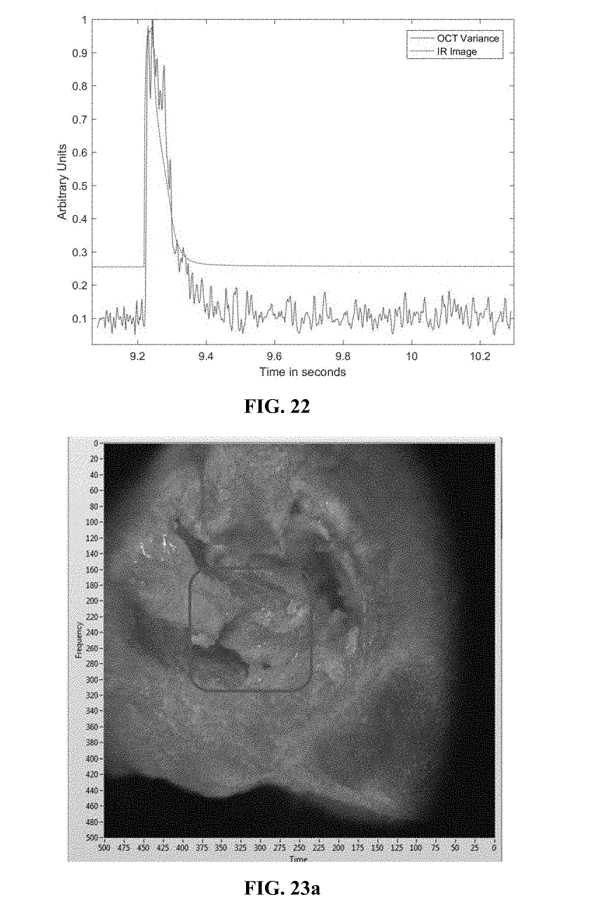

FIGS. 20a-20d demonstrate coagulation in the mouse brain in vivo coupled with laser irradiation. FIG. 20a shows a bloodflow overlay in jet color format on the native OCT cross-section image of the mouse brain showing the process of coagulation with laser irradiation, where the brightness of the blood flow contrast goes down as laser irradiation is carried out over a specific location of the blood vessel, as shown in FIG. 20b. FIG. 20c shows a bloodflow overlay in jet color format on the native OCT cross-section image of the mouse brain showing the process of coagulation with laser irradiation, where the brightness of the blood flow contrast goes down as laser irradiation is carried out over a specific location of the blood vessel, as shown in FIG. 20d.

### EXAMPLE

### Side Cut/Skin Applications Setup

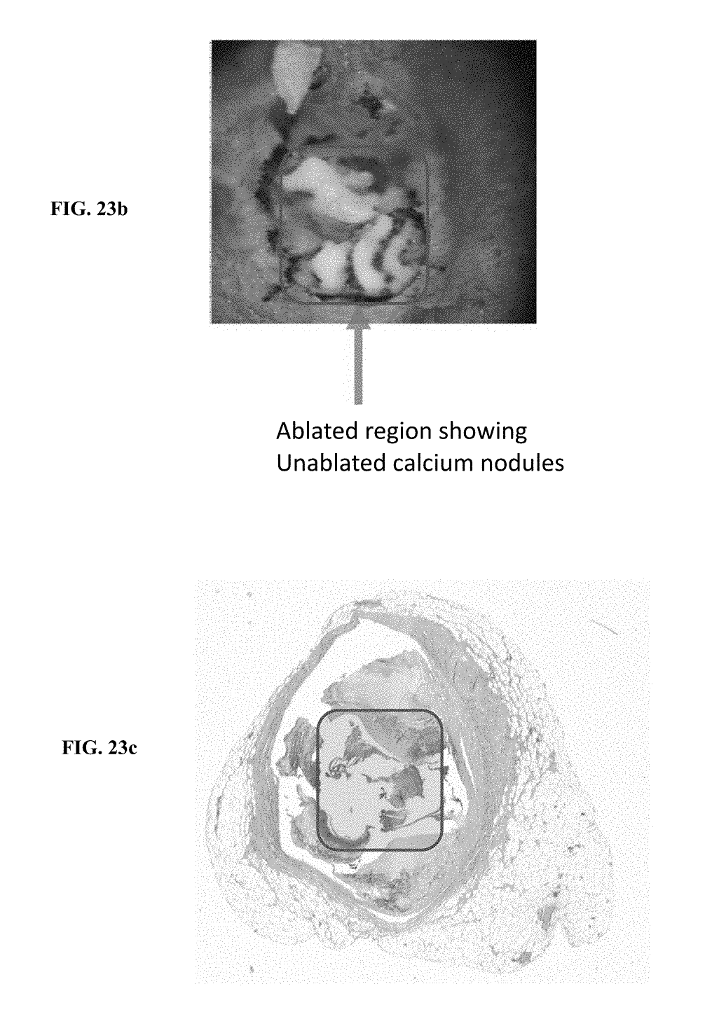

FIG. 21a is a y-z OCT image before skin being cut, and FIG. 21b is a y-z OCT image after the skin has been cut. FIG. 21c is an x-z OCT image before being cut, and FIG. 21d is an x-z OCT image after the skin has been cut.

### EXAMPLE

### OCT Thermography for the Image Guided System

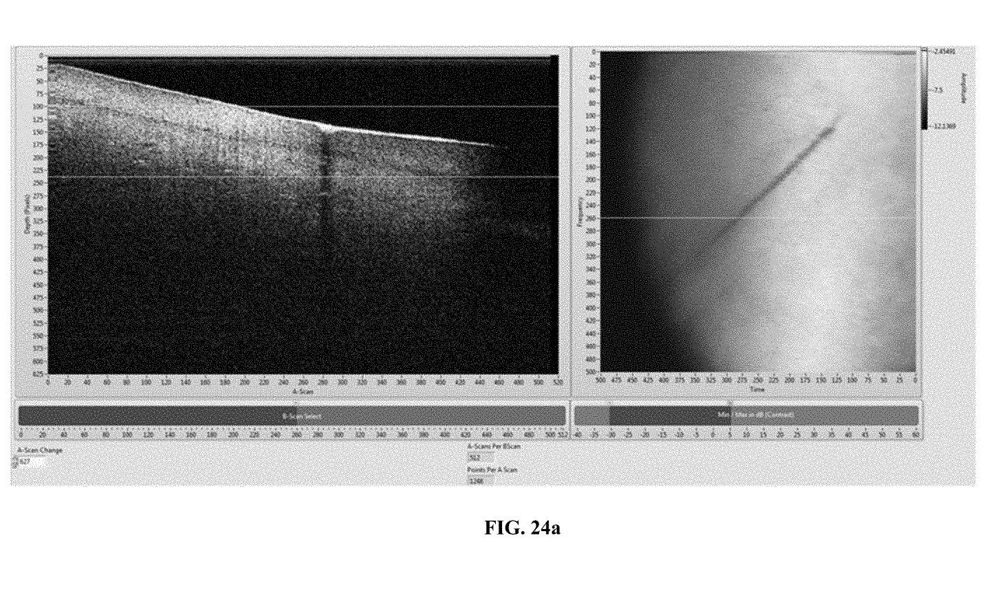

OCT provides image information (e.g., angiogram and optical properties) for feedback to control the surgical laser based on the tissues thermal reaction to the surgical laser. The OCT results verified with a PDMS sample and an IR camera as shown in FIG. 22.

### EXAMPLE

### Chronic Total Occlusion (CTO) or Coronary Artery Ablation

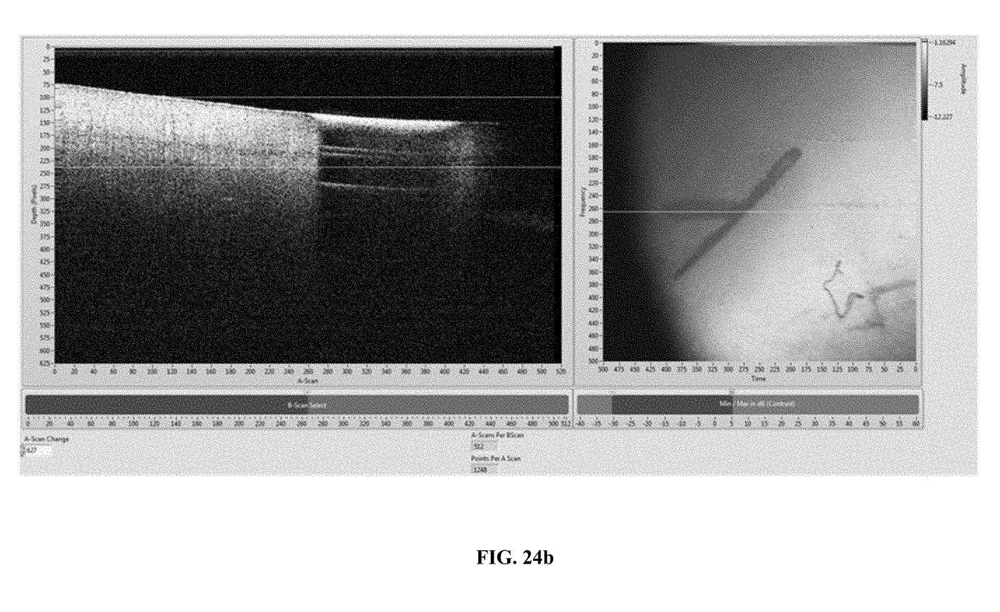

A coronary artery that was totally occluded was removed from a human cadaver. The removed artery was cut such that the plane of the cut was perpendicular to the long axis of the artery, thereby exposing the occlusion for imaging, as shown in FIG. 23a. Optical Coherence Tomography images were taken of the occluded artery before ablation. A co-aligned Thulium laser was used to ablate a square region of the occlusion. OCT imaging was performed during ablation as feedback and after ablation to assess the ablated region. The ablated artery was subsequently examined histologically. As shown in FIG. 23c, histology confirms that the occlusion was heterogeneous, consisting of calcium and softer tissue. Although the softer tissue was ablated, the calcium nodules were not ablated at the laser energy levels used in the experiment, as shown in FIG. 23b. Subsequent experiments have shown that calcium rich CTOs can also be completely ablated using higher laser energy levels.

FIG. 23a is an OCT Image of Occluded Artery Before Ablation; FIG. 23b is an OCT Image of Occluded Artery After Ablation, where the square highlights the Ablated region showing Unablated calcium nodules; and FIG. 23c is an Histology Image of Occluded Artery Before Ablation.

### EXAMPLE

### LASER/OCT Cartilage Experiments

Cartilage is made of 80% water. Currently used laser are 1.5 um Er-Doped. 1.94 um will have a better scope of usage in these techniques. OCT provides better depth feedback than current tech of reflected intensity.

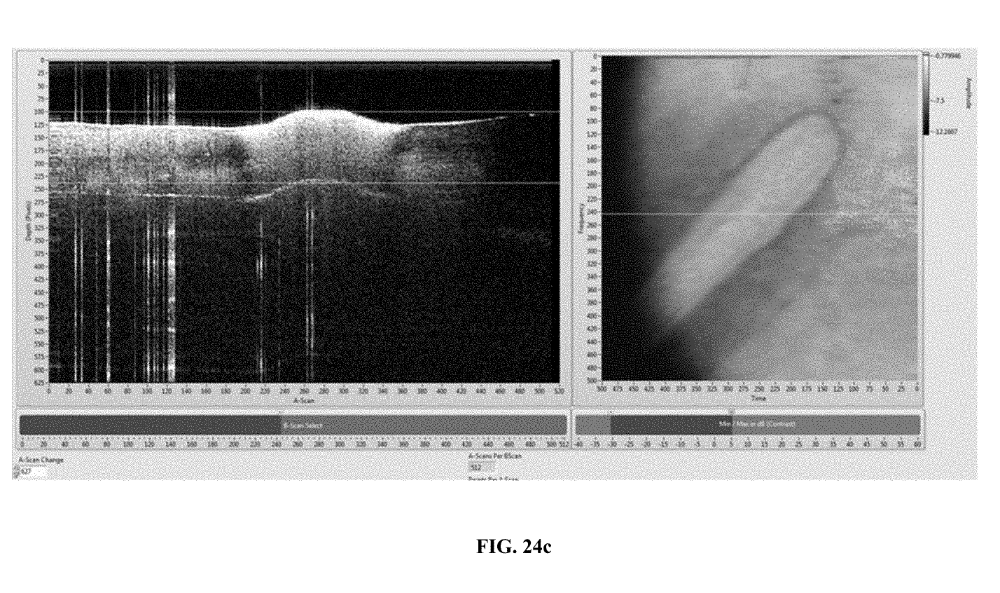

Mechanism of laser-induced tissue regeneration includes: creating a plurality of micro-pores in cartilage matrix promote water permeability and increase the feeding of biological cells and dynamic mechanical oscillations activate tissue regeneration. FIG. 24a is an OCT image of the cartilage after 1 Tm laser sweep. FIG. 24b is an OCT image of the cartilage after multiple Tm laser sweeps showing the micropore. And FIG. 24c is an OCT image of the cartilage after 100 Tm laser passes showing the increased diameter of the micropore.

Computer Implemented Component or Systems

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, systems may include program modules, which may include routines, programs, components, data structures, etc. that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

In some embodiments, some or all of the functions or process(es) described herein and performed by a computer program that is formed from computer readable program code and that is embodied in a computer readable medium. The phrase “computer readable program code” includes any type of computer code, including source code, object code, executable code, firmware, software, etc. The phrase “computer readable medium” includes any type of medium capable of being accessed by a computer, such as read only memory (ROM), random access memory (RAM), a hard disk drive, a compact disc (CD), a digital video disc (DVD), or any other type of memory.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An image guided system comprising:
an Optical Coherence Tomography (OCT) imaging system to provide high resolution, three-dimensional image information;
providing an OCT image of a diseased area and a non-diseased area surrounding the tissue; and
a surgical tool for a treatment of the diseased area.

2. The system of claim 1, wherein the surgical tool is a laser system including a pulse energy, a laser pulse duration, a pulse repetition rate, a spot size, and a laser emission wavelength; and the laser minimizes non-specific damage to non-diseased area surrounding the tissue through the OCT imaging system.

3. The image guided system of claim 2, wherein the OCT image is computed using OCT processing techniques include acquiring the spectral fringe signal to two-byte values, applying a Hanning window, computing a fast Fourier transform and a resultant power spectrum vs. time delay of light propagating into the diseased area and the non-diseased area surrounding the tissue.

4. The image guided system of claim 3, wherein the OCT imaging system controls the lateral positions of the laser system by a motor control and controls an average power level, wherein the laser system includes a plurality of profiles of a laser turn-ON time and a laser turn-OFF time that avoided non-diseased area surrounding the tissue and stored in a computer-readable media based on the OCT images including an A-scan location.

5. The image guided system of claim 4, wherein the image guided system approves a proposed ablation pattern to initiate tissue removal by reading the computer-readable media and turning on the laser system at the appropriate A-scan locations during imaging of a next OCT image frame so as to avoid non-diseased area surrounding the tissue through 2D image processing

6. The image guided system of claim 4, wherein the 2D image processing includes an Edge/Flow detection and an ablation profile generation.

7. An image guided system comprising
a Combined Holistic Surgical View subsystem operably coupled to a Feature Detection Image Overlay subsystem, an examination system operably coupled to the Feature Detection Image Overlay subsystem, a Positioning subsystem operably coupled with an examination system, and a Treatment system operably coupled with the Positioning subsystem;
the Combined Holistic Surgical View subsystem includes an imaging system for preoperative imaging and intraoperative imaging, where the imaging system combines the preoperative imaging and intraoperative imaging into one holistic view of a surgical field, and the imaging system provides a high resolution volume OCT image;
the Feature Detection Image Overlay subsystem analyzes the OCT volume image, highlights features of surgical relevance, and overlays the OCT volume image on the holistic view;
the examination system conducts an examination to determine where to position a surgical instrument, and the examination system performs the examination and acquires secondary OCT volume images by the examination system interacting with the feature detection overlay system to highlight structural features;
the Positioning subsystem includes the examination system coupled with the combined holistic view and a highlight of structural features, and the Positioning subsystem positions the surgical instrument within an x,y,z location of the surgical field that is constantly tracked by the imaging system to detail the surgical instrument's position within the surgical field and integrates new OCT image data into the combined holistic surgical view; and
the treatment system executes a treatment on the tissue and is operably coupled with the imaging system to acquire OCT images simultaneously with the treatment.

8. The image guided system of claim 7, wherein the treatment is a laser treatment and treatment system controls laser dosimetry and laser energy.

9. The image guided system of claim 8, wherein the treatment system is a robotic treatment system.

10. The image guided system of claim 8, wherein the treatment includes a myocardial infarct, the Feature Detection Image Overlay subsystem detects blood vessels, ischemic tissue, and the sites for microwell incision; the treatment system drives the laser to laser-cut microwells into the epicardium while avoiding unwanted damage to the vascular sites and, the treatment system includes an injector to deposit angiogenic chemokines to penetrate the myocardial infarct at the microwells; and the vascular sites is sealed with the tomographic image guidance controlling the co-aligned tissue modifying laser.

11. The image guided system of claim 8, wherein the treatment is cancer, the treatment system provides highly localized chemotherapeutic or radiological-seed treatment to cancer margins as tumor tissue is imaged and classified in vivo, the image-guided system includes tomographic image guidance to use a laser to cut tissue and inject these chemotherapeutic or radiological-seed treatment, and seal the tissue with laser modification of the surface of the tissue.

12. The image guided system of claim 8, wherein the treatment is damaged articular cartilage, where tomographic imaging reveals damaged cartilage tissue, the image guided system then deposits autologous stem cells into microwells; the treatment system includes multiplexing cell laden hydro-gel with normal hydro-gel to ensure no cross-contamination and a secondary conduit in the image guided system transfers the solvent location in order to seal the cartilage.

13. An image guided system comprising an OCT imaging system operably coupled with a laser system, wherein the OCT Imaging system includes an OCT source and the OCT source is a swept-source mode-locked laser source, a sample arm and a reference arm, where backscattered light from the reference arm and sample arm interfere to form a fringe.

14. The image guide system of claim 13, wherein the swept-source mode-locked laser source is centered at about 1310 nm±70 nm, with a fast scan-rate of about 100 kHz; and the sample arm and the reference arm are path length and dispersion matched.

15. The image guided system of claim 14, wherein the laser system is a nanosecond pulsed fiber laser system used for cutting the tissue and the laser system co-aligns a cutting laser beam with an OCT beam to produce a combined laser/OCT beam.

16. The image guided system of claim 15, further comprising a Biomaterial/Cell deposition system operably coupled with the laser system to deposits a material onto a modified tissue.

17. The image guided system of claim 16, wherein the Biomaterial/Cell deposition system is loaded with a cell-seeded-polymer and a cross-linking agent to form a hydrogel deposition.

18. The image guided system of claim 17, wherein the OCT system positions Biomaterial/Cell deposition system to a micro-well in the tissue and the OCT system adjusts location of subsequent images to capture the hydrogel deposition.

19. The image guided system of claim 18, wherein the Biomaterial/Cell deposition system includes an applicator-optical mount that interfaces directly with an optical table mount and the Biomaterial/Cell deposition system where the hydrogel deposition can be imaged without adjustment.

20. The image guided system of claim 8, wherein the laser coagulates the tissue and then the laser removes the tissue.
